(19) United States
(12) Patent Application Publication
(10) Pub. No.: US 2026/0077089 A1
(43) Pub. Date: Mar. 19, 2026

(54) Biopolymers for Use in Wound Care

(71) Applicant: ExoPolymer, Inc., San Carlos, CA (US)

(72) Inventor: Derek Wells, Palo Alto, CA (US)

(73) Assignee: ExoPolymer, Inc., Mountain View, CA (US)

(21) Appl. No.: 19/109,474

(22) PCT Filed: Sep. 13, 2023
(86) PCT No.: PCT/US2023/074060

§ 371 Date: Mar. 6, 2025

Related U.S. Application Data
(60) Provisional application No. 63/406,670, filed on Sep. 14, 2022.

(51) Int. Cl. A61L 26/00; A61L 15/28; A61L 15/44; A61L 15/60

(52) U.S. Cl. CPC A61L 26/0023; A61L 15/28; A61L 15/44; A61L 15/60; A61L 26/0066; A61L 2300/232; A61L 2300/236

(57) ABSTRACT

Provided herein are biopolymers comprising repeating polysaccharide units, preparations of biopolymers, and compositions and dressings comprising biopolymers, as well as methods of use.

### CROSS-REFERENCE TO RELATED APPLICATIONS

This application claims the benefit of priority of U.S. Provisional Application No. 63/406,670, filed Sep. 14, 2022, which is incorporated by reference herein in its entirety for any purpose.

### FIELD

Provided herein are biopolymers comprising repeating polysaccharide units, preparations of biopolymers, and compositions and dressings comprising biopolymers, as well as methods of use.

### BACKGROUND

Despite advances in moist wound care, wounds still remain a major cause of morbidity and mortality and are a key driver of global health care costs. It is estimated that 2.5% of the US Population suffers from chronic wounds at an estimated cost which ranges between $28.1-96.8 billion dollars. Less economic data is available for acute wounds, however data suggests that acute wounds accounted for 17.2 million hospital visits in 2014. In addition, wounds negatively impact quality of life for millions of people and more importantly, have a very high correlation to mortality. Diabetic foot ulcers, for example, have a 5-year mortality rate of 31% which is comparable to that of cancers.

Polymers that bind and retain water have important therapeutic value in wound care due to increased hydration and moisture content which maintains a moist wound environment. Unlike traditional gauze or cloth bandages, skin dressings containing water-binding polymers can both sequester fluids and keep tissue moist. A moist wound environment results in a number of benefits which lead to more rapid and higher quality wound healing. These benefits include autolytic debridement and reduced pain and scarring. This is accomplished through increased collagen synthesis, keratinocyte migration and increased presence of nutrients, growth factors and other beneficial factors in the wound environment.

A wide range of water-binding polymers, biologically and synthetically derived, are used for both acute and chronic moist wound care. At the most basic level, these products capture fluid exudate from the injury, create a moist environment for healing to occur and form a physical barrier against additional insults to the wound surface. In more advanced polymer systems, additional components may be added. Further improvements to wound care include both polymers and polymer-membrane systems which allow for the timed release of therapeutic agents or the sequestration of specific materials that are essential for microbial growth.

Moist wound dressings incorporate a range of water-binding technologies to accomplish the goal of wound care. These include biopolymers such as agar, alginates, collagens and pectin and semi-synthetic polymers including carboxymethyl cellulose and fully synthetic polymers such as acrylates or polyvinyl alcohols. Combinations of these materials have also been used.

An alternative biopolymer product that shows improved moisture binding, and improved efficacy in wound care would have significant benefit for both individual patients and society at large.

There are many water binding polymers used in wound care including, but not limited to, agar, alginae, carageenan, locus bean gum, guar gum, tragacanth, gum arabic, xanthan, karaya, tara gum, gellan, pectin, chitosan, hyaluronic acid, modified starch, cellulose, cellulose ethers (e.g. carboxymethylcellulose, hydroxypropylcellulose, hydroxypropylmethylcellulose, methylcellulose and methylethylcellulose), acrylates, polyvinyl alcohols.

Despite a wide array of polymers available for wound care, there are still unmet patient needs which could be addressed by new water binding molecules.

### SUMMARY

The present disclosure provides naturally produced, non-animal-derived carbohydrate biopolymers having improved capabilities, as compositions and dressings that are particularly suitable for therapeutic wound care, comprising such biopolymers, as well as their use in therapeutic wound care. As shown in the Examples provided herein, biopolymers of the present disclosure exhibit exceptionally high moisture binding capacity. Biopolymers of the present disclosure are particularly suited to wound care and lack cytotoxicity in standardized testing. Furthermore, the biopolymers of the present disclosure, when used as functional ingredients in a dressing, maintain a moist wound environment and show improved wound healing.

Biopolymers of the present disclosure are not derived from animal sources and can be made through fermentation of non-pathogenic microbes using agricultural feedstocks.
Embodiment 1. A dressing comprising a biopolymer preparation comprising a biopolymer, wherein the biopolymer is composed of repeating disaccharide units comprising glucose and galactose, wherein at least 60%, at least 65%, at least 70%, at least 75%, at least 80%, at least 85%, or at least 90% of the glucose is acetylated, and wherein at least 40%, at least 45%, at least 50%, at least 55%, at least 60%, at least 65%, at least 70%, at least 75%, at least 80%, at least 85%, at least 90%, at least 95%, or at least 99% of the galactose is pyruvylated, and wherein the biopolymer preparation is incorporated on or in a solid substrate.
Embodiment 2. The dressing of embodiment 1, wherein the glucose and galactose are linked by β-1,3 glycosidic bonds and α-1,3 glycosidic bonds.
Embodiment 3. A dressing comprising a biopolymer preparation comprising a biopolymer that is composed of repeating disaccharide units of the structure:

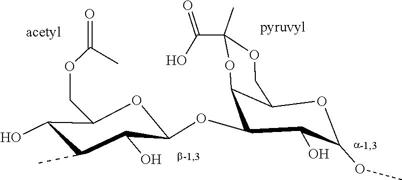

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the disaccharide units lack the pyruvyl moiety, and wherein the biopolymer preparation is incorporated on or in a solid substrate.
Embodiment 4. The dressing of any one of embodiments 1-3, wherein the molar ratio of glucose:galactose:pyruvyl:acetyl in the biopolymer is 1:1:0.4-1:0.6-1.
Embodiment 5. The dressing of any one of embodiments 1-4, wherein the average molecular weight of the biopolymer in the biopolymer preparation is less than 3,000 kDa, less than 1,000 kDa, less than 300 kDa, less than 100 kDa, or less than 40 kDa.
Embodiment 6. The dressing of any one of embodiments 1-4, wherein the average molecular weight of the biopolymer in the biopolymer preparation is 0.5 kDa to 40 kDa.
Embodiment 7. The dressing of any one of the preceding embodiments, wherein the biopolymer preparation is capable of absorbing at least the same amount, at least 1.5-fold, at least 2-fold, or at least 3-fold more water than an equal amount of hyaluronic acid.
Embodiment 8. The dressing of any one of the preceding embodiments, wherein the biopolymer preparation is capable of absorbing an amount of water that is at least 100%, at least 200%, at least 300%, at least 400%, or at least 500% the initial dry weight of the biopolymer preparation.
Embodiment 9. The dressing of embodiment 7 or embodiment 8 wherein water absorption is measured by placing a dry sample of the biopolymer preparation in a humidified chamber at 30° C. for five days.
Embodiment 10. The dressing of any one of the preceding embodiments, wherein the biopolymer preparation is less than 25%, less than 20%, less than 15%, less than 10%, less than 5%, less than 3%, or less than 1% succinoglycan, or is substantially free of succinoglycan.
Embodiment 11. The dressing of any one of the preceding embodiments, wherein the biopolymer preparation is a powder or rehydrated powder.
Embodiment 12. A dressing comprising a biopolymer preparation comprising a biopolymer that is composed of repeating disaccharide units comprising glucose and galactose, wherein at least 60%, at least 65%, at least 70%, at least 75%, at least 80%, at least 85%, or at least 90% of the glucose is acetylated, and wherein less than 15%, less than 10%, or less than 5% of the galactose is pyruvylated, and wherein the biopolymer preparation is incorporated on or in a solid substrate.
Embodiment 13. The dressing of embodiment 12, wherein the glucose and galactose are linked by β-1,3 glycosidic bonds and α-1,3 glycosidic bonds.
Embodiment 14. A dressing comprising a biopolymer preparation, wherein the biopolymer is composed of repeating disaccharide units of the structure:

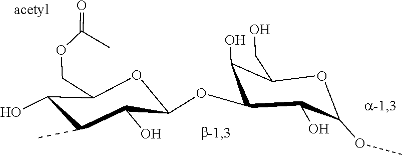

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 15%, no more than 10%, or no more than 5%, of the disaccharide units are pyruvylated, and wherein the biopolymer preparation is incorporated on or in a solid substrate.
Embodiment 15. The dressing of any one of embodiments 12-14, wherein the molar ratio of glucose:galactose:pyruvyl:acetyl in the biopolymer is 1:1:<0.5:0.6-1.
Embodiment 16. The dressing of any one of embodiments 12-15, wherein the average molecular weight of the biopolymer in the biopolymer preparation is less than 3,000 kDa, less than 1,000 kDa, less than 300 kDa, less than 100 kDa, or less than 40 kDa.
Embodiment 17. The dressing of any one of embodiments 12-15, wherein the average molecular weight of the biopolymer in the biopolymer preparation is 0.5 kDa to 40 kDa.
Embodiment 18. The dressing of any one of embodiments 12-17, wherein the biopolymer preparation is capable of absorbing at least the same amount, at least 1.1-fold, at least 1.2-fold, at least 1.3-fold, at least 1.4-fold, or at least 1.5-fold more water than an equal amount of hyaluronic acid.
Embodiment 19. The dressing of any one of embodiments 12-18, wherein the biopolymer preparation is capable of absorbing an amount of water that is at least 100%, at least 200%, at least 300%, or at least 400% the initial dry weight of the biopolymer preparation.
Embodiment 20. The dressing of embodiment 18 or embodiment 19, wherein water absorption is measured by placing a dry sample of the biopolymer preparation in a humidified chamber at 30° C. for five days.
Embodiment 21. The dressing of any one of embodiments 12-20, wherein the biopolymer preparation is less than 25%, less than 20%, less than 15%, less than 10%, less than 5%, less than 3%, or less than 1% succinoglycan.
Embodiment 22. The dressing of any one of embodiments 12-21, wherein the biopolymer preparation is a powder or rehydrated powder.
Embodiment 23. A dressing comprising a biopolymer preparation comprising a biopolymer that is composed of repeating polysaccharide units of the structure:

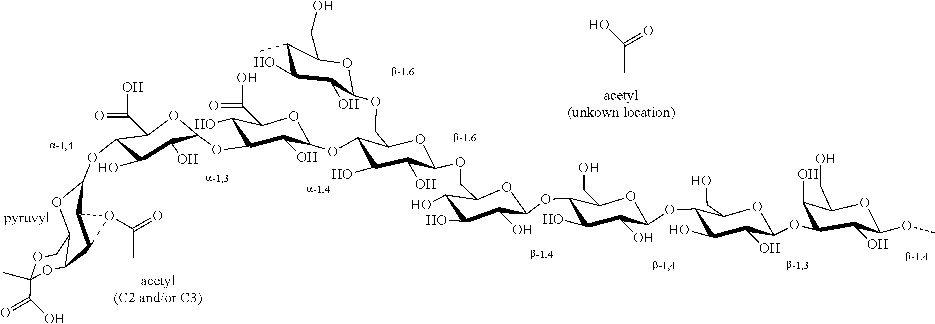

wherein the dotted lines represent the bonds between polysaccharide units; wherein no more than 30%, no more than 25%, no more than 20%, no more than 15%, no more than 10%, no more than 5%, or no more than 1% of the polysaccharide units lack an acetyl moiety; and wherein no more than 10%, no more than 5%, or no more than 1% of the polysaccharide units lack the pyruvyl moiety, and wherein the biopolymer preparation is incorporated on or in a solid substrate.
Embodiment 24. The dressing of embodiment 23, wherein the average molecular weight of the biopolymer in the biopolymer preparation is less than 3,000 kDa, less than 1,000 kDa, less than 300 kDa, less than 100 kDa, or less than 40 kDa.
Embodiment 25. The dressing of embodiment 23, wherein the average molecular weight of the biopolymer in the biopolymer preparation is 1.6 kDa to 40 kDa.
Embodiment 26. The dressing of any one of embodiments 23-25, wherein the biopolymer preparation is capable of absorbing at least the same amount, at least 1.1-fold, at least 1.2-fold, at least 1.3-fold, at least 1.4-fold, or at least 1.5-fold more water than an equal amount of hyaluronic acid.
Embodiment 27. The dressing of any one of embodiments 23-26, wherein the biopolymer preparation is capable of absorbing an amount of water that is at least 100%, at least 200%, at least 300%, or at least 400% the initial dry weight of the biopolymer preparation.
Embodiment 28. The dressing of embodiment 26 or embodiment 27, wherein water absorption is measured by placing a dry sample of the biopolymer preparation in a humidified chamber at 30° C. for five days.
Embodiment 29. The dressing of any one of embodiments 23-28, wherein the biopolymer preparation is a powder or rehydrated powder.
Embodiment 30. A dressing comprising a biopolymer preparation comprising a biopolymer that is composed of repeating polysaccharide units, wherein each polysaccharide unit comprises 2-15 or 2-12 or 2-10 monosaccharides, and wherein the biopolymer has a negative charge:monosaccharide ratio in the repeating polysaccharide unit of at least 0.3, or at least 0.35, or at least 0.4, or at least 0.45, and wherein the biopolymer preparation is incorporated on or in a solid substrate.
Embodiment 31. The dressing of embodiment 30, wherein the polysaccharide unit comprises at least one galactose linked to at least one glucose.
Embodiment 32. The dressing of embodiment 31, wherein at least one galactose is linked to a glucose through a β-1,3 glycosidic bond.
Embodiment 33. The dressing of any one of embodiments 30-32, wherein the average molecular weight of the biopolymer in the biopolymer preparation is less than 3,000 kDa, less than 1,000 kDa, less than 300 kDa, less than 100 kDa, or less than 40 kDa.
Embodiment 34. The dressing of any one of embodiments 30-32, wherein the average molecular weight of the biopolymer in the biopolymer preparation is 0.5 kDa to 40 kDa.
Embodiment 35. The dressing of any one of embodiments 30-34, wherein the biopolymer preparation is capable of absorbing at least the same amount, at least 1.1-fold, at least 1.5-fold, at least 2-fold, or at least 3-fold more water than an equal amount of hyaluronic acid.
Embodiment 36. The dressing of any one of embodiments 30-35, wherein the biopolymer preparation is capable of absorbing an amount of water that is at least 100%, at least 200%, at least 300%, or at least 400% the initial dry weight of the biopolymer preparation.
Embodiment 37. The dressing of embodiment 35 or embodiment 36, wherein water absorption is measured by placing a dry sample of the biopolymer preparation in a humidified chamber at 30° C. for five days.
Embodiment 38. The dressing of any one of embodiments 30-37, wherein the biopolymer preparation is a powder or rehydrated powder.
Embodiment 39. The dressing of any one of the preceding embodiments, wherein the dressing is a wound dressing.
Embodiment 40. The dressing of any one of the preceding embodiments, wherein the dressing is skin dressing.
Embodiment 41. The dressing of any one of the preceding embodiments, wherein the dressing is a bandage.
Embodiment 42. The dressing of any one of the preceding embodiments, wherein the biopolymer preparation forms one layer of the dressing.
Embodiment 43. The dressing of any one of the preceding embodiments, wherein the solid substrate comprises woven materials of natural or synthetic fibers or synthetic polymeric sheets.
Embodiment 44. The dressing of any one of the preceding embodiments further comprising an adhesive material.
Embodiment 45. The dressing of any one of the preceding embodiments further comprising one or more additional ingredients selected from the group consisting of an additional hydrocolloid agent, alcohol, peroxide, betadine, sequestration agents, antimicrobials, antibacterials, antivirals, antifungals, anti-inflammatories, wound healing promoters, conditional agents, surfactants, anti-scaring medications, analgesics, anesthetics, and steroids.
Embodiment 46. The dressing of embodiment 45, wherein the hydrocolloid agents are selected from the group consisting of collagen, cellulose and cellulose derivatives, glycosaminoglycans, pectins, gum arabic, galactomannans, agar, carrageen, alginates, gelatin, caseinates, xanthans, dextrans, scleroglucans, and combinations thereof.
Embodiment 47. The dressing of embodiment 45 or embodiment 46, wherein the dressing comprises hyaluronic acid.
Embodiment 48. The dressing of embodiment 45 or embodiment 46, wherein the dressing does not comprise hyaluronic acid.
Embodiment 49. The dressing of any one of embodiments 45-48, wherein the dressing comprises silver.
Embodiment 50. The dressing of any one of embodiments 45-49, wherein the one or more additional ingredients are incorporated into the layer comprising the biopolymer preparation, into the adhesive material, or into one or more additional layers.
Embodiment 51. The dressing of any one of the preceding embodiments, wherein the biopolymer preparation is dispersed throughout a hydrophilic polyurethane foam matrix, and the hydrophilic polyurethane foam matrix is incorporated on or in the solid substrate.
Embodiment 52. The dressing of any one of the preceding embodiments, wherein the dressing is a multi-layered dressing.
Embodiment 53. A method of treating a wound in a subject in need thereof, comprising applying the dressing of any one of the preceding embodiments to the wound.
Embodiment 54. The method of embodiment 53, wherein the wound comprises an acute wound, a chronic wound, a necrotizing wound, or an infected wound.
Embodiment 55. The method of embodiment 53 or embodiment 54, wherein the wound is a mechanical wound, a thermal wound, a chemical wound, or a radiation wound.
Embodiment 56. The method of any one of embodiments 53-55, wherein the dressing is applied to the face, ears, forehead, neck, arms, upper chest, legs, feet, and/or hands of the subject.
Embodiment 57. The method of any of embodiments 53-56, wherein the dressing promotes thermal insulation of damaged organ, tissue, or skin, absorption of excess exudate and moisture, protection against exogenous infections, and optimum moisture in the wound environment at the interface between wound and dressing.
Embodiment 58. A method of treating a wound in a subject in need thereof, comprising applying a composition to the wound, wherein the composition comprises a biopolymer, wherein the biopolymer is composed of repeating disaccharide units comprising glucose and galactose, wherein at least 60%, at least 65%, at least 70%, at least 75%, at least 80%, at least 85%, or at least 90% of the glucose is acetylated, and wherein at least 40%, at least 45%, at least 50%, at least 55%, at least 60%, at least 65%, at least 70%, at least 75%, at least 80%, at least 85%, at least 90%, at least 95%, or at least 99% of the galactose is pyruvylated.
Embodiment 59. The method of embodiment 58, wherein the glucose and galactose are linked by β-1,3 glycosidic bonds and α-1,3 glycosidic bonds.
Embodiment 60. A method of treating a wound in a subject in need thereof, comprising applying a composition to the wound, wherein the composition comprises a biopolymer, wherein the biopolymer is composed of repeating disaccharide units of the structure:

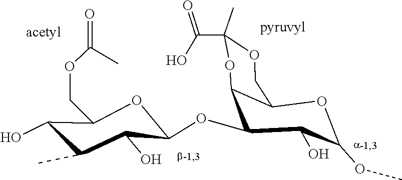

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the disaccharide units lack the pyruvyl moiety.
Embodiment 61. The method of any one of embodiments 58-60, wherein the molar ratio of glucose:galactose:pyruvyl:acetyl in the biopolymer is 1:1:0.4-1:0.6-1.
Embodiment 62. The method of any one of embodiments 58-61, wherein the average molecular weight of the biopolymer in the composition is less than 3,000 kDa, less than 1,000 kDa, less than 300 kDa, less than 100 kDa, or less than 40 kDa.
Embodiment 63. The method of any one of embodiments 58-61, wherein the average molecular weight of the biopolymer in the composition is 0.5 kDa to 40 kDa.
Embodiment 64. The method of any one of embodiments 58-63, wherein the biopolymer is capable of absorbing at least the same amount, at least 1.5-fold, at least 2-fold, or at least 3-fold more water than an equal amount of hyaluronic acid.
Embodiment 65. The method of any one of embodiments 58-64, wherein the biopolymer is capable of absorbing an amount of water that is at least 100%, at least 200%, at least 300%, at least 400%, or at least 500% the initial dry weight of the biopolymer.
Embodiment 66. The method of embodiment 64 or embodiment 65, wherein water absorption is measured by placing a dry sample of the biopolymer in a humidified chamber at 30° C. for five days.
Embodiment 67. The method of any one of embodiments 58-66, which is substantially free of succinoglycan.
Embodiment 68. A method of treating a wound in a subject in need thereof, comprising applying a composition to the wound, wherein the composition comprises a biopolymer, wherein the biopolymer is composed of repeating disaccharide units comprising glucose and galactose, wherein at least 60%, at least 65%, at least 70%, at least 75%, at least 80%, at least 85%, or at least 90% of the glucose is acetylated, and wherein less than 15%, less than 10%, or less than 5% of the galactose is pyruvylated.
Embodiment 69. The method of embodiment 68, wherein the glucose and galactose are linked by β-1,3 linkages and α-1,3 linkages.
Embodiment 70. A method of treating a wound in a subject in need thereof, comprising applying a composition to the wound, wherein the composition comprises a biopolymer, wherein the biopolymer is composed of repeating disaccharide units of the structure:

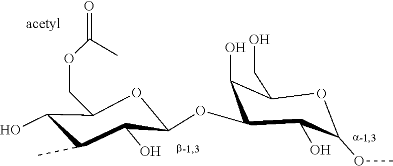

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 15%, no more than 10%, or no more than 5%, of the disaccharide units are pyruvylated.
Embodiment 71. The method of any one of embodiments 68-70, wherein the molar ratio of glucose:galactose:pyruvyl:acetyl in the biopolymer is 1:1:<0.5:0.6-1.
Embodiment 72. The method of any one of embodiments 68-71, wherein the average molecular weight of the biopolymer in the composition is less than 3,000 kDa, less than 1,000 kDa, less than 300 kDa, less than 100 kDa, or less than 40 kDa.
Embodiment 73. The method of any one of embodiments 68-71, wherein the average molecular weight of the biopolymer in the composition is 0.5 kDa to 40 kDa.
Embodiment 74. The method of any one of embodiments 68-73, wherein the composition is capable of absorbing at least the same amount, at least 1.1-fold, at least 1.2-fold, at least 1.3-fold, at least 1.4-fold, or at least 1.5-fold more water than an equal amount of hyaluronic acid.
Embodiment 75. The method of any one of embodiments 68-74, wherein the composition is capable absorbing an amount of water that is at least 100%, at least 200%, at least 300%, or at least 400% the initial dry weight of the biopolymer.
Embodiment 76. The method of embodiment 74 or embodiment 75, wherein water absorption is measured by placing a dry sample of the biopolymer in a humidified chamber at 30° C. for five days.
Embodiment 77. The method of any one of embodiments 68-76, wherein the composition comprises the biopolymer preparation of any one of embodiments 12-22.
Embodiment 78. The method of any one of embodiments 68-77, which is substantially free of succinoglycan.
Embodiment 79. A method of treating a wound in a subject in need thereof, comprising applying a composition to the wound, wherein the composition comprises a biopolymer, wherein the biopolymer is composed of repeating polysaccharide units of the structure:

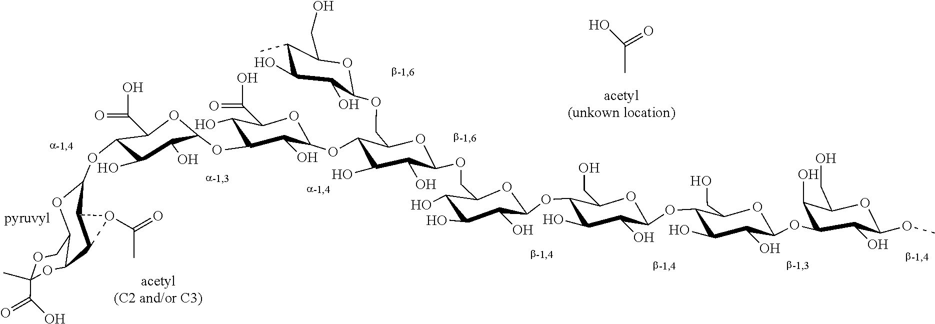

wherein the dotted lines represent the bonds between polysaccharide units; wherein no more than 30%, no more than 25%, no more than 20%, no more than 15%, no more than 10%, no more than 5%, or no more than 1% of the polysaccharide units lack an acetyl moiety; and wherein no more than 10%, no more than 5%, or no more than 1% of the polysaccharide units lack the pyruvyl moiety.
Embodiment 80. The method of embodiment 79, wherein the average molecular weight of the biopolymer in the composition is less than 3,000 kDa, less than 1,000 kDa, less than 300 kDa, less than 100 kDa, or less than 40 kDa.
Embodiment 81. The method of embodiment 79, wherein the average molecular weight of the biopolymer in the composition is 1.6 kDa to 40 kDa.
Embodiment 82. The method of any one of embodiments 79-81, wherein the composition is capable of absorbing at least the same amount, at least 1.1-fold, at least 1.2-fold, at least 1.3-fold, at least 1.4-fold, or at least 1.5-fold more water than an equal amount of hyaluronic acid.
Embodiment 83. The method of any one of embodiments 79-82, wherein the composition is capable of absorbing an amount of water that is at least 100%, at least 200%, at least 300%, or at least 400% the initial dry weight of the biopolymer.
Embodiment 84. The method of embodiment 82 or embodiment 83, wherein water absorption is measured by placing a dry sample of the biopolymer in a humidified chamber at 30° C. for five days.
Embodiment 85. The method of any one of embodiments 79-84, wherein the composition comprises the biopolymer preparation of any one of embodiments 23-39.
Embodiment 86. A method of treating a wound in a subject in need thereof, comprising applying a composition to the wound, wherein the composition comprises a biopolymer, wherein the biopolymer is composed of repeating polysaccharide units, wherein each polysaccharide unit comprises 2-15 or 2-12 or 2-10 monosaccharides, and wherein the biopolymer has a negative charge:monosaccharide ratio in the repeating polysaccharide unit of at least 0.3, or at least 0.35, or at least 0.4, or at least 0.45.
Embodiment 87. The method of embodiment 86, wherein the polysaccharide unit comprises at least one galactose linked to at least one glucose.
Embodiment 88. The method of embodiment 87, wherein at least one galactose is linked to a glucose through a β1,3 glycosidic bond.
Embodiment 89. The method of any one of embodiments 86-88, wherein the average molecular weight of the biopolymer in the composition is less than 3,000 kDa, less than 1,000 kDa, less than 300 kDa, less than 100 kDa, or less than 40 kDa.
Embodiment 90. The method of any one of embodiments 86-88, wherein the average molecular weight of the biopolymer in the composition is 0.5 kDa to 40 kDa.
Embodiment 91. The method of any one of embodiments 86-90, wherein the composition is capable of absorbing at least the same amount, at least 1.1-fold, at least 1.5-fold, at least 2-fold, or at least 3-fold more water than an equal amount of hyaluronic acid.
Embodiment 92. The method of any one of embodiments 86-91, wherein the composition is capable of absorbing an amount of water that is at least equal to, at least 1.5 times, at least two times, at least 2.5 times, at least three times, at least four times, or at least five times the initial dry weight of the biopolymer.
Embodiment 93. The method of embodiment 91 or embodiment 92, wherein water absorption is measured by placing a dry sample of the biopolymer in a humidified chamber at 30° C. for five days.
Embodiment 94. The method of any one of embodiments 86-93, wherein the composition comprises the biopolymer preparation of any one of embodiments 30-38.
Embodiment 95. The method of any one of embodiments 58-94, wherein the composition further comprising one or more additional ingredients selected from the group consisting of an additional hydrocolloid agent, alcohol, peroxide, betadine, sequestration agents, antimicrobials, antibacterials, antivirals, antifungals, anti-inflammatories, wound healing promoters, conditional agents, surfactants, anti-scaring medications, analgesics, anesthetics, and steroids.
Embodiment 96. The method of embodiment 95, wherein the hydrocolloid agents are selected from the group consisting of collagen, cellulose and cellulose derivatives, glycosaminoglycans, pectins, gum arabic, galactomannans, agar, carrageen, alginates, gelatin, caseinates, xanthans, dextrans, scleroglucans, and combinations thereof.
Embodiment 97. The method of embodiment 95 or embodiment 96, wherein the composition comprises hyaluronic acid.
Embodiment 98. The method of embodiment 95 or embodiment 96, wherein the composition does not comprise hyaluronic acid.
Embodiment 99. The method of any one of embodiments 95-98, wherein the composition comprises silver.
Embodiment 100. The method of any one of embodiments 58-99, wherein the composition is a salve, emulsion, cream, milk, ointment, syndet, solution, serum, gel, spray or aerosol, foam, suspension, lotion, or stick.
Embodiment 101. The method of embodiment 100, wherein the composition is incorporated on or in a solid substrate in the form of a dressing.
Embodiment 102. The method of embodiment 101, wherein the solid substrate comprises woven materials of natural or synthetic fibers or synthetic polymeric sheets.
Embodiment 103. The method of any one of embodiments 101-102, wherein the dressing further comprising an adhesive material.
Embodiment 104. The method of any one of embodiments 101-103, wherein the dressing is a wound dressing.
Embodiment 105. The method of any one of embodiments 101-104, wherein the dressing is skin dressing.
Embodiment 106. The method of any one of embodiments 101-105, wherein the dressing is a bandage.
Embodiment 107. The method of any one of embodiments 101-106, wherein the composition forms one layer of the dressing.
Embodiment 108. The method of embodiment 107, wherein the one or more additional ingredients are incorporated into the layer comprising the biopolymer preparation, in the adhesive material, or in one or more additional layers.
Embodiment 109. The method of any one of embodiments 101-108, wherein the dressing is a multi-layered dressing.
Embodiment 110. The method of embodiment 109, wherein the multi-layered dressing comprises 1) an inner layer forming the wound contacting surface of the dressing and adhering the dressing to the wound, comprising the biopolymer, and 2) an outer cover layer.
Embodiment 111. The method of embodiment 110, wherein the multi-layered dressing further comprises additional layer(s) between the inner layer and the outer cover layer.
Embodiment 112. The method of any one of embodiments 58-111, wherein the wound comprises an acute wound, a chronic wound, a necrotizing wound, or an infected wound.
Embodiment 113. The method of any of embodiments 58-112, wherein the wound is a mechanical wound, a thermal wound, a chemical wound, or a radiation wound.
Embodiment 114. The method of any of embodiments 58-113, wherein the dressing promotes thermal insulation of damaged organ, tissue, or skin, absorption of excess exudate and moisture, protection against exogenous infections, and optimum moisture in the wound environment at the interface between wound and dressing.

### BRIEF DESCRIPTION OF THE DRAWINGS

FIG. 1. Representative repeating unit of a polysaccharide biopolymer. Individual sugar residues are linked by glycosidic bonds. Polysaccharide-based biopolymers consist of a main chain of sugar residues that are connected by glycosidic linkages. The main chain may also have a side chain consisting of sugar residues, and each may have different chemical modifications. The main and side chains of a biopolymer, along with chemical modifications, make up a repeating unit. Repeating units are connected by glycosidic linkages to generate longer polymers.

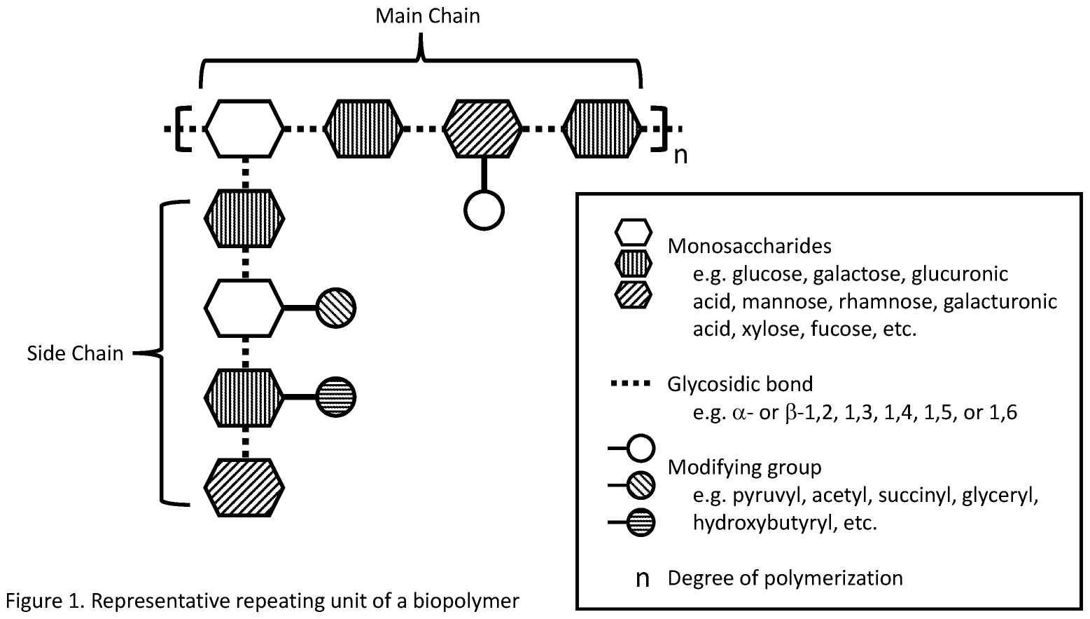

FIG. 2. Biopolymer structures. Galactoglucan is a repeating dimer of galactose and glucose, with pyruvyl and acetyl modifications. Succinoglycan is a repeating octamer of one galactose and seven glucose residues, with pyruvyl, acetyl, and succinyl modifications. Glucuronoglycan is a repeating nonamer of two galactose, two glucuronic acid, and five glucose residues with pyruvyl and acetyl modifications.

FIG. 3. Water absorption of isolated and mixed biopolymers compared to HA. The ability to bind water, as measured by mass increase, was determined for individual and mixed biopolymers. Mass increase for each biopolymer was divided by the mass increase of hyaluronic acid (HA) to derive relative fold change in water retention.

FIG. 4. Correlation between negative charge and water absorption. The charge of each biopolymer was calculated at physiological pH based on pKa of the acidic modifying group or sugar acid. This value was then divided by the number of sugar residues in a repeating unit to generate a ratio for each biopolymer. Charge density was plotted against the percent mass increase of each biopolymer.

FIG. 5. Water absorption of non-pyruvylated galactoglucan compared to HA. The ability to bind water was measured as described in Example 4 and FIG. 3. Mass increase was normalized to the value for HA, and fold change was calculated.

FIG. 6. Cytotoxicity of biopolymers. Cytotoxicity testing was performed according to the ISO 10993-5 standard. Biopolymers were dissolved in PBS at a concentration of 1% (w/v), heat pasteurized, and used in cytotoxicity assays. Values for each biopolymer were compared to both positive and negative controls.

FIG. 7. Transdermal Penetration and Skin Retention Assay Results. Unidirectional permeability across ex-vivo human skin tissue was determined using Franz Cells. Results are shown for the amount of carbohydrate in a representative receptor chamber (permeate) after 4 hours of treatment with a donor solution of 5% (w/v) of non-pyruvylated galactoglucan. After completion of the Franz Cell Assay, the skin sample was rinsed and then soaked in PBS to elute carbohydrate that was absorbed or associated with the skin surface. Total carbohydrate in receptor solution or in eluted skin sample was determined using the anthrone assay. Background levels from control receptor chambers and skin samples were subtracted to derive the reported values.

### DETAILED DESCRIPTION

The present disclosure provides naturally produced, non-animal-derived carbohydrate biopolymers having moisture binding capabilities. The present disclosure also provides a composition comprising the biopolymers discussed herein, dressing comprising such composition, and use of the composition and dressing for therapeutic wound care.

A wound is understood to mean a break in the continuity of body tissues with or without substance loss.

Wounds are classified into various types depending on their causes. Thus, wounds created by external trauma are classed as mechanical wounds, these mainly being cutting and piercing wounds, crushing, laceration, scratch and abrasion wounds. Another form of wounds is described as thermal wounds, which are caused by the action of extreme heat or cold. In contrast, chemical wounds are caused by the action of chemicals, in particular by erosion by acids or alkalis. Tissue breaks or damage which arise under the action of actinic radiation, e.g. ultraviolet radiation and/or ionizing radiation, are described as radiation wounds. Depending on the physiological condition, a distinction is also made between necrotizing, infected and/or chronic or acute wounds. A wound is considered chronic when its healing is not completed within a period of four to eight weeks after occurrence.

Wound care or wound treatment generally pursues the aim of preventing a wound infection and ensuring rapid and effective wound healing.

The term “dressing” as used herein refers to a dressing for application onto a wound or injury in order to prevent contact with and/or penetration of foreign substances, stop bleeding, and/or promote tissue healing. In some embodiments, the dressing is applied to an organ. In some embodiments, the dressing is a skin dressing for topical application onto skin. In some embodiments, the dressing is a wound dressing.

The term “wound dressing” as used herein refers to dressings for application onto internal or external wounds, in order to prevent penetration of foreign substances into the wound and to absorb blood and wound secretions. According to the invention, terms such as “wound plaster”, “wound bandage” or “wound covering” can also be used.

Natural product biopolymers with improved moisture binding properties, that are low molecular weight (LMW) and produced by the fermentation of non-pathogenic microbes, are attractive in wound healing for a number of different reasons. First, such biopolymers are cost-effective to produce and can be blended in medical and wound care products, such as skin dressings, to achieve a moist wound environment and improved wound healing performance. Second, these biopolymers can be added to a wide range of products where hydration provides improved performance, such as compositions or dressings in wound care. Third, a biopolymer that is produced by microbial fermentation is an attractive alternative to animal-derived products and mitigates social and/or moral issues associated with animal use. Biopolymers from harmless microbes, as opposed to pathogenic ones, require fewer downstream processing steps to manufacture a safe product.

The present invention provides compositions of biopolymers based on exopolysaccharides produced by non-pathogenic species of soil bacteria, dressings comprising such compositions, and pharmaceutical use of such biopolymers in wound healing. High water binding capacity is a desirable property of a dressing for wound care as this helps to maintain a moist environment. Biocompatibility is also essential in wound care. Provided herein are isolated biopolymers that have substantially improved water binding properties in comparison to hyaluronic acid. In some embodiments, the isolated biopolymers are derived from Rhizobiaceae bacteria. These bacteria naturally produce low molecular weight biopolymers, which have the ability to penetrate the skin. Manufacture of these biopolymers by fermentation is highly advantaged relative to the processes for other wound care biopolymers.

The present inventors have identified certain biopolymers that have superior water retention properties, and are thus ideal for use as dressings in wound care. The first molecule, galactoglucan, is a repeating disaccharide of galactose and glucose with pyruvyl and acetyl modifications. It may be derived from several different species of Rhizobiaceae, including the bacterium Sinorhizobium meliloti (aka Ensifer meliloti) as well as other closely related species and is naturally produced at low molecular weight (LMW) during fermentation. It is one of two biopolymers that are naturally produced by this organism, and its moisture binding properties can only be observed independently of the other biopolymer, succinoglycan. We have demonstrated that galactoglucan has greater than 3.5× the water retention capacity of HA. A variant of galactoglucan, which lacks the pyruvyl modification, shows 1.7× the water retention capacity of HA. The second molecule, glucuronoglycan, is a repeating nonasaccharide containing galactose, glucuronic acid, and glucose with pyruvyl and acetyl modifications. It may be derived from several different species of Rhizobiaceae, including the bacterium Sinorhizobium fredi (aka Ensifer fredii) and is naturally produced as a mixture of high and low molecular weights. We have demonstrated that glucuronoglycan has 1.7× the water retention capacity of HA.

In addition to the increased water retention performance, galactoglucan and glucuronoglycan are non-toxic in a standard pre-clinical model, a prerequisite for suitability of the compounds in dermatological uses, such as compositions or dressing in wound care. In some embodiments, these LMW biopolymer fractions penetrate through skin samples in an in vitro Franz cell assay. In dressing formulations, in some embodiments, the inclusion of Rhizobiaceae-derived biopolymers in dressing, such as wound dressing, provides improved moist wound environment and thus, improved wound healing.

This invention provides microbial polymers with beneficial performance features—moisture retention—for use as beneficial ingredients in dressing formulations to take up or bind wound fluids and also serve as moisture buffer wherein excess water or excess moisture can be temporarily stored and as necessary released, to maintain a moist wound environment during the treatment and/or healing of the wound. These microbial biopolymers show substantially improved moisture retention in comparison to a different active ingredient, hyaluronic acid, which is used in wound healing. The Rhizobiaceae-derived biopolymers described herein are suitable for use in dressing formulations, and can provide increased wound healing in dressing products. Further, the production of biopolymers by fermentation, requiring little downstream processing, provides an economically advantaged method of production compared to incumbent technologies for the manufacture of animal- or microbial-derived biopolymers.

### Biopolymers and Biopolymer Preparations

The Rhizobiaceae, a family of soil-dwelling, symbiotic bacteria, have been studied for decades for their ability to provide fixed nitrogen to their leguminous plant hosts, but to date have not been fully exploited as fermentative microorganisms for the production of bioindustrial, pharmaceutical, or cosmetic products. These bacteria naturally produce water-soluble exopolysaccharides, or biopolymers, which have roles in both host plant association and biofilm formation. The variety of exopolysaccharides produced by the Rhizobiaceae suggests a breadth of novel biopolymers with new functionalities that could add substantial value to several markets.

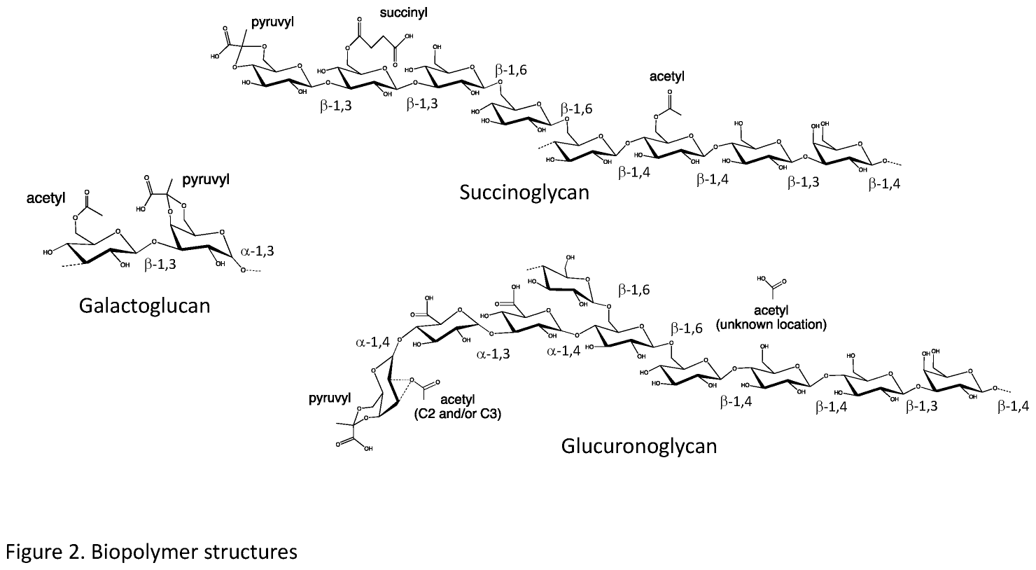

Succinoglycan and Galactoglucan Sinorhizobium (Ensifer) meliloti naturally produces two acidic exopolysaccharides: succinoglycan (EPS 1), and galactoglucan (EPS II) (Barnett 2018). Succinoglycan is the major exopolysaccharide produced by S. meliloti. The repeating unit of succinoglycan (FIG. 2) consists of glucose and galactose in a 7:1 ratio with acetyl, pyruvyl and succinyl modifications (Reuber 1993). Succinoglycan is naturally produced by S. meliloti at both high and low molecular weights. The general mechanism of succinoglycan biosynthesis is relatively well understood and likely shared by related organisms that produce similar biopolymers.

Galactoglucan production is restricted to species that are phylogenetically close to S. meliloti. The repeating unit of galactoglucan (FIG. 2) consists of glucose and galactose in a 1:1 ratio with acetyl and pyruvyl modifications (Glazebrook 1989). Specific linkages are β-D-Glcp-(1-3)-α-D-Galp-(1-3), with a 6-O-acetyl on most D-glucose residues, and a 4,6-O-pyruvyl on every D-galactose (Her 1990). Galactoglucan is naturally produced by S. meliloti at low molecular weights. In comparison to succinoglycan, relatively little is known about the biosynthetic pathway of galactoglucan.

Glucuronoglycan Sinorhizobium (Ensifer)fredii naturally produces an acidic exopolysaccharide that consists of glucose, galactose, and glucuronic acid in a 5:2:2 ratio with acetyl and pyruvyl modifications (Djordjevic 1986) (FIG. 2). The biopolymer depicted in FIG. 2 is produced by several S. fredii type strains including NGR234, HH103 (ATCC51809), and likely USDA257 (Gray 1991, Pueppke 1999, Rodriguez-Navarro 2014). According to structural analyses, the terminal galactose on the side chain of glucuronoglycan is 4,6-pyruvylated, and it can be acetylated on either the second, third, or both carbons of the same sugar residue. A third acetyl group has also been detected in glucuronoglycan from the HH103 strain, and therefore likely the other strains as well, but its location has not been elucidated (Staehelin 2006, Rodriguez-Navarro 2014). The exo region of these S. fredii species spans 28 kb and shares a high degree of synteny with the S. meliloti cluster responsible for succinoglycan biosynthesis. Many of the genes share homology with the S. meliloti exo genes (Zhan 1990). Glucuronoglycan has a similar structure to succinoglycan, but contains glucuronic acid and is not succinylated. The main chain of glucuronoglycan consists of six sugar residues, whereas that of succinoglycan contains four. Glucuronoglycan is produced by S. fredii strains at both high and low molecular weights (Staehelin 2006).

In some embodiments, a biopolymer is provided that is composed of repeating disaccharide units comprising glucose and galactose, wherein at least 60%, at least 65%, at least 70%, at least 75%, at least 80%, at least 85%, or at least 90% of the glucose is acetylated, and wherein at least 40%, at least 45%, at least 50%, at least 55%, at least 60%, at least 65%, at least 70%, at least 75%, at least 80%, at least 85%, at least 90%, at least 95%, or at least 99% of the galactose is pyruvylated. In some embodiments, the glucose and galactose are linked by β-1,3 glycosidic bonds and α-1,3 glycosidic bonds. In some embodiments, a biopolymer is provided that is composed of repeating disaccharide units of the structure:

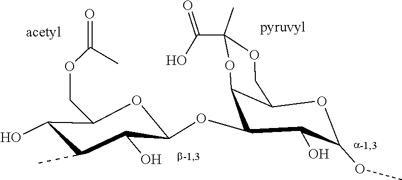

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the disaccharide units lack the pyruvyl moiety. In some embodiments, the biopolymer is comprised in a biopolymer preparation. In some such embodiments, the molar ratio of glucose:galactose:pyruvyl:acetyl in the biopolymer preparation is 1:1:0.4-1:0.6-1. In some embodiments, a preparation of the biopolymer is provided that comprises less than 25%, less than 20%, less than 15%, less than 10%, less than 5%, less than 3%, or less than 1% succinoglycan. In some embodiments, a preparation of the biopolymer is substantially free of succinoglycan.

In some embodiments, a biopolymer is provided that is composed of repeating disaccharide units comprising glucose and galactose, wherein at least 60%, at least 65%, at least 70%, at least 75%, at least 80%, at least 85%, or at least 90% of the glucose is acetylated, and wherein less than 15%, less than 10%, or less than 5% of the galactose is pyruvylated. In some embodiments, the glucose and galactose are linked by β-1,3 glycosidic bonds and α-1,3 glycosidic bonds. In some embodiments, a biopolymer is provided that is composed of repeating disaccharide units of the structure:

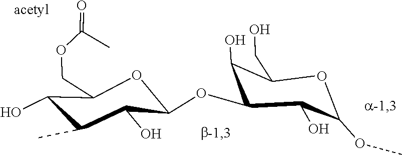

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 15%, no more than 10%, or no more than 5%, of the disaccharide units are pyruvylated. In some embodiments, the biopolymer is comprised in a biopolymer preparation. In some such embodiments, the molar ratio of glucose:galactose:pyruvyl:acetyl in the biopolymer preparation is 1:1:<0.5:0.6-1. In some embodiments, a preparation of the biopolymer is provided that comprises less than 25%, less than 20%, less than 15%, less than 10%, less than 5%, less than 3%, or less than 1% succinoglycan. In some embodiments, a preparation of the biopolymer is substantially free of succinoglycan.

In some embodiments, a biopolymer is provided, wherein the biopolymer is composed of repeating polysaccharide units of the structure:

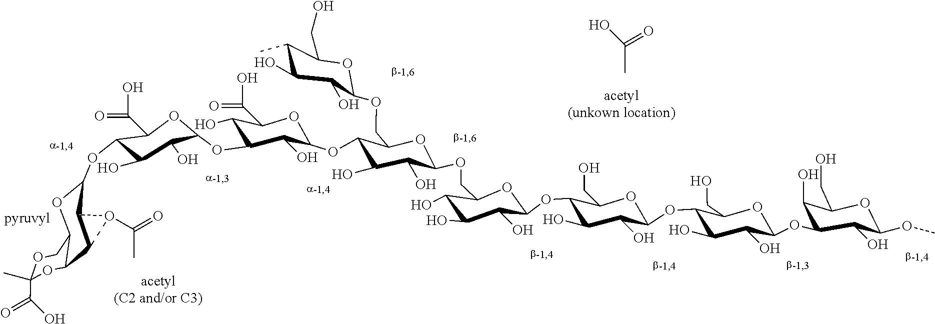

wherein the dotted lines represent the bonds between polysaccharide units; wherein no more than 30%, no more than 25%, no more than 20%, no more than 15%, no more than 10%, no more than 5%, or no more than 1% of the polysaccharide units lack an acetyl moiety; and wherein no more than 10%, no more than 5%, or no more than 1% of the disaccharide units lack the pyruvyl moiety. In some embodiments, a polysaccharide unit comprises one, two, are three acetyl groups. In some embodiments, the average number of acetyl groups per polysaccharide unit is 1-3.

In some embodiments, a biopolymer is provided, wherein the biopolymer is composed of repeating polysaccharide units, wherein each polysaccharide unit comprises 2-15 or 2-12 or 2-10 monosaccharides, and wherein the biopolymer has a negative charge:monosaccharide ratio in the repeating polysaccharide unit of at least 0.3, or at least 0.35, or at least 0.4, or at least 0.45. In some embodiments, the polysaccharide unit comprises at least one galactose linked to at least one glucose. In some such embodiments, at least one galactose is linked to a glucose through a β-1,3 glycosidic bond.

In various embodiments, the average molecular weight of the biopolymer in the biopolymer preparation is less than 3,000 kDa, less than 1,000 kDa, less than 300 kDa, less than 100 kDa, or less than 40 kDa. In some embodiments, the average molecular weight of the biopolymer in the biopolymer preparation is 0.5 kDa to 40 kDa or 1.6 kDa to 40 kDa.

In some embodiments, the biopolymer is capable of absorbing at least the same amount, at least 1.1-fold, at least 1.2-fold, at least 1.3-fold, at least 1.4-fold, 1.5-fold, at least 2-fold, or at least 3-fold more water than an equal amount of hyaluronic acid. In some embodiments, the biopolymer is capable of absorbing an amount of water that is at least 100%, at least 200%, at least 300%, at least 400%, or at least 500% the initial dry weight of the biopolymer preparation. Water absorption may be measured, for example, by placing a dry sample of the biopolymer or a biopolymer preparation in a humidified chamber at an approximately constant temperature for a fixed length of time. In some embodiments, the temperature is about 20° C., about 25° C., about 30° C., about 35° C., or about 37° C. In some embodiments, the fixed length of time is one day, two days, three days, four days, five days, six days, or one week.

### Strain Construction

Both S. meliloti and S. fredii are amenable to genetic modification, and a common method for strain engineering is to use homologous recombination, antibiotic resistance, and sucrose counter selection (Quandt 1993) to delete specific regions in the genome. Plasmids that contain modified genomic regions can be constructed and then used to replace native regions with targeted changes. By introduction of these non-replicating plasmids by conjugal transfer, strains with single integrations can be selected by antibiotic resistance and confirmed by PCR. Secondarily, integrated plasmids can be counter selected due the presence of the sacB gene, which encodes a levansucrase that is lethal to Gram negative bacteria in the presence of sucrose. Antibiotic sensitive, sucrose resistant strains will then either have recombined to wild type, or have incorporated a deletion, insertion, or other modification that was present in the constructed plasmid. Modified strains can be confirmed by PCR and sequencing.

Unmodified, non-domesticated strains of S. meliloti produce both succinoglycan and galactoglucan, and are suitable for the simultaneous production of both biopolymers. In certain type strains, such as Rm1021 (ATCC51124), the ability to produce galactoglucan has been lost due to lab strain domestication (Charoenpanich 2015). In the case of domesticated strains, there are several methods by which a galactoglucan producing strain can be constructed. Examples include restoration of an intact expR gene, knock out of mucR, overexpression of WggR (Bahlawane 2008), or growth in phosphate-limited medium (Mendrygal 2000).

For the production of succinoglycan in the absence of galactoglucan, the type strain Rm1021 can be used. There are several regulatory genes which can be modified resulting in strains which overproduce succinoglycan. These genes include exoR, exoS, chvI, syrM, and nodD3 (Barnett 2015). Others include syrA, mucR (Keller 1995), and exoX (Zhan 1990). If a non-domesticated strain of S. meliloti is used, it is necessary to knock out galactoglucan biosynthetic genes to generate a strain that only produces succinoglycan. The genes required for galactoglucan biosynthesis fall within a 32 kb region of pSymB and include six predicted glycosyltransferases and four genes predicted to encode proteins required for the synthesis of dTDP-glucose and dTDP-rhamnose (Becker 1997). Any of several glycosyltransferases, such as wgaB or wgeB may be excised in order to eliminate production of galactoglucan.

To produce galactoglucan in the absence of succinoglycan, wild type strains of S. meliloti with mutations in succinoglycan biosynthetic genes can be generated using pJQ200SK. Additionally, domesticated strains with the restored ability to produce galactoglucan, via any of the methods described above, may be used. The biosynthetic cluster specific for succinoglycan is located within a 22 kb region on pSymB. Structural and regulatory roles have been assigned to several of the genes in this cluster (Reuber 1993). To eliminate succinoglycan biosynthesis, any of several genes, such as exoA, exoF, exoL, exoM, exoP, exoQ, exoT, or exoY, may be excised genetically.

Glucuronoglycan is the major product of wild type S. fredii, and no modifications of strains are necessary for the production of this biopolymer. Any of the strains mentioned above may be used for production and further analysis of material.

### Methods of Making Biopolymers

For production of biopolymers, several different liquid growth media can be used. S. meliloti strains grow well on LB or TY medium, and these can be supplemented with an additional carbon source such as glucose, sucrose, or succinate to boost production of product. S. fredii can be grown on TY medium, and supplementation with additional carbon source is beneficial to production. Both S. meliloti and S. fredii can be grown on defined minimal medium, such as M9 or MOPS-mannitol, which can result in higher yields. Minimal medium allows for precise control over fermentation variables such as phosphate concentration, pH, micronutrients, sulfate concentration, and carbon source.

Alcohol precipitation may be used to purify biopolymers after fermentation. Typically, cells are removed from fermentation broth by centrifugation or filtration. High viscosity of fermentation broth may necessitate the addition of one to two volumes of water to assist in cell separation procedures. To further remove residual cells or cell debris, the cell-free supernatant may be incubated with protease. To precipitate biopolymer, isopropanol or ethanol, as well as a mono- or divalent cation such as KCl or CaCl₂ in a concentration range around 1 mM, can be added to the cell-free supernatant, typically at 1× to 2× the culture volume. Biopolymers precipitate upon mixing, and can be isolated by centrifugation or filtration. Further purification steps may be undertaken at this point to reduce salt concentrations or any cell debris that may have precipitated with the polymer. These steps may include additional alcohol washes, protease treatments, rehydration, centrifugation, dialysis, solvent washes, lyophilization, etc., that suit the desired end use. Purified product can be dried in an oven until mass stabilizes (all unbound water has evaporated). Dried product can be ground, milled, or otherwise processed to generate final, purified biopolymers.

### Wound Care Compositions and Dressings

In various embodiments, compositions are provided, comprising the biopolymer(s) provided herein for treating a wound and to assist in the wound healing process.

In various embodiments, the compositions comprise one, two, or three of the biopolymers provided herein. In various embodiments, the composition comprises 1-99% w/v of the biopolymer(s) provided herein, or any amount therebetween. By way of example, the amount of biopolymer may be from 10%-90%, from 10%-80%, from 10%-70%, 10%-60%, 20%-90%, 20%-80%, 20%-70%, 20%-60%, 30%-90%, 30%-80%, 30%-70%, 30%-60%, 40%-90%, 40%-80%, 40%-70%, 40%-60%, 50-90%, 50-80%, 50-70%, 60%-90%, 60%-80%, 70%-90%, etc.

In various embodiments, the compositions may be in aqueous solution, alcohol, or oil-in-water emulsion, in the form of salves, emulsions, creams, milks, ointments, syndets, solutions, sera, gels, sprays or aerosols, foams, suspensions, lotions, or sticks.

In some embodiments, the compositions are incorporated onto or embedded in a dressing. In some embodiments, the dressings are for wound care (and may be referred to as “wound dressings”). The biopolymer(s) provided herein may be coated onto and/or embedded in a solid substrate to produce a dressing suitable for application on internal or external wounds. In some embodiments, the dressings are skin dressings, and the skin dressing is suitable for topical application onto skin, such as wounded skin.

In various embodiments the solid substrate means a flexible material that is capable of (1) supporting and retaining a biopolymer composition/preparation discussed herein applied into and/or within a surface of the flexible material, (2) conforming to a wound surface of a subject (human or animal) with which it comes into contact, and (3) maintaining the contact with the subject's wound surface for an extended period of time, so as to facilitate application of the composition to the wound surface.

In various embodiments, the solid substrate material is not particularly limited as long as it can provide a suitable substrate for the biopolymer and are sufficiently strong to withstand removal from the wound surface or skin of a subject and maintain its integrity during use.

Suitable solid substrate material is exemplified by woven materials comprising natural fibers and/or synthetic fibers. Suitable natural fibers are exemplified by cotton fibers, linen fibers, hemp fibers and the like. Suitable synthetic fibers comprise filaments formed from polymers exemplified by polyamides, polyesters, polystyrenes, polyacrylates, vinyl polymers (e.g., polyethylene, polytetrafluoro-ethylene, polypropylene and polyvinyl chloride), polycarbonates, polyurethanes, poly dimethyl siloxanes, cellulose acetates, polymethyl methacrylates, ethylene vinyl acetates, polysulfones, nitrocelluloses and similar copolymers. Suitable polymers also include biological polymers which can be naturally occurring or produced in vitro by fermentation and the like. Suitable biological polymers are exemplified by collagen, elastin, silk, keratin, and copolymers thereof. The woven materials may be gauze-like in that they may be very thin and loosely woven. Alternatively, the woven materials may more tightly woven from thicker fibers.

Alternatively, suitable solid substrate materials are exemplified by synthetic polymeric sheets comprising materials exemplified by polyethylene films, polypropylene films, polyamide films and the like. Particularly suitable are liquid-impermeable, vapor-permeable membranes allow vapors to egress from a wound site through the material while preventing liquids from ingressing or egressing through the material. Such membranes are commonly referred to as high moisture vapor transmission rate (MVTR) films formed from hydrophilic polymers exemplified by polyvinyl alcohol, polyvinyl acetate, cellulose-based materials (e.g., ethers, esters, nitrates, and the like) polyvinyl pyrrolidone, polyurethanes, polyamides, polyesters, polyacrylates, polymethacrylates, and polyacrylamides. Such MVTR fims may be cross-linked, or blended, or grafted, or copolymerized with each other.

In some embodiments, the wound dressing comprising the solid substrate and the at least one exemplary biopolymer of the present disclosure, also optionally has an adhesive material to facilitate adherence of the wound dressing in contact with a target wound site after the wound dressing is applied in place. A wide range of adhesive materials can be used for the dressing, and can be selected to maximize adhesion, absorption and comfort, while minimizing irritation to the user. The adhesive layer is preferably efficient at adhering to, but not damaging to the dermis or wound site. The adhesive layer further preferably has a relatively greater adherence to the film than to the dermis or wound site. There can be a desired range of adhesive strength for the adhesive layer in the present invention. The strength can vary relative to the selected use of the dressing.

In some embodiments, the dressing comprises one, two, or three of the biopolymers provided herein, and the biopolymer(s) form one layer of the dressing. In some embodiments, the layer comprising the biopolymer provided herein comprises 1-99% w/v of the composition, or any amount therebetween. By way of example, the amount of biopolymer may be from 10%-90%, from 10%-80%, from 10%-70%, 10%-60%, 20%-90%, 20%-80%, 20%-70%, 20%-60%, 30%-90%, 30%-80%, 30%-70%, 30%-60%, 40%-90%, 40%-80%, 40%-70%, 40%-60%, 50-90%, 50-80%, 50-70%, 60%-90%, 60%-80%, 70%-90%, etc. In some embodiments, the composition comprising the biopolymer may be formulated as a powder, and subsequently rehydrated. The composition is then incorporated on or in the dressing.

In some embodiments, the solid substrate material is a flexible film layer and extends beyond the layer comprising the biopolymer(s).

One skilled in the art can select additional suitable ingredients for the composition or dressing. In some embodiments, the composition or dressing can include one or more of the following additional ingredients: additional hydrocolloid agent(s); those which can assist with wound protection and healing, such as alcohol, peroxide or betadine; sequestration agents, such as avidin; antimicrobials, such as silver; antibacterials, such as Triclosan, or polysporin; antivirals, such as Nonoxyl-9; antifungals, such as imidazole; antinflamatories such as hydrocortizone; wound healing promoters, such as growth factors; collagen; conditional agents; surfactants; anti-scaring medications such as cortisone or pharmacologically active agents, including, but not limited to, analgesics, anesthetics, anti-inflammatories, and steroids. Hydrocolloid agents can be selected from the group consisting of collagen, cellulose and cellulose derivatives, glycosaminoglycans (in particular acidic glycosamino-glycans, preferably hyaluronic acid and/or salts thereof), pectins, gum arabic, galactomannans, agar, carrageen, alginates, gelatin, caseinates, xanthans, dextrans and scleroglucans, or combinations thereof. Conditional agents can be selected from the group consisting of vitamins, mineral salts, trace elements, plant extracts, animal extracts, proteins, enzymes, vitamin E, vitamin A, aloe, and combinations thereof. The antimicrobial compositions can include silver; aminoglycosides exemplified by tobramycin, gentamicin, neomycin, streptomycin, and the like; azoles exemplified by fluconazole, itraconazole, and the like; β-lactam antibiotics exemplified by penams, cephems, carbapenems, monobactams, β-lactamase inhibitors, and the like; cephalosporins exemplified by cefacetrile, cefadroxyl, cephalexin, cephazolin, cefproxil, cefbuperazone, and the like; chloramphenicol; clindamycin; fusidic acid; glycopeptides exemplified by vancomycin, teicoplanin, ramoplanin, and the like; macrolides exemplified by azithromycin, clarithromycin, dirithromysin, erythromycin, spiramycin, tylosin, and the like; metronidazole; mupirocin; penicillins exemplified by benzylpenicillin, procaine benzylpenicillin, benzathine benzylpenicillin, phenoxymethylpenicillin, and the like; polyenes exemplified by amphotericin B, nystatin, natamycin, and the like; quinolones exemplified by ciprofloxacin, ofloxacin, danofloxacin, and the like; rifamycins exemplified by rifampicin, rifabutin, rifapentine, rifaximin, and the like; sufonamides exemplified by sulfacetamine, sulfadoxine, and the like; tetracyclines exemplified by doxycycline, minocycline, tigecycline, and the like; and trimethoprim, among others.

In some embodiments, the composition or dressing comprises at least one biopolymer provided herein and hyaluronic acid.

In some embodiments, the additional ingredients can be incorporated on and/or in the layer of the dressing comprising the biopolymer(s) provided herein, the adhesive material of the dressing, or both. The additional ingredients may also alternatively be incorporated into one or more additional layers of the dressing.

One example of the dressing comprises a solid substrate and a hydrophilic polyurethane foam matrix affixed to the solid substrate, wherein the hydrophilic polyurethane foam matrix has at least one biopolymer provided herein dispersed throughout said hydrophilic polyurethane foam matrix. The dressing is formed by polymerizing an aqueous mixture having at least one biopolymer provided herein with an aqueous mixture of a hydrophilic polyurethane prepolymer, in a predetermined ratio, so that the polymerization of the polyurethane foam forms a matrix binder for the at least one biopolymer provided herein.

In some embodiments, the dressing is a multi-layered wound dressing, with each layer performing a different function. For example, the multi-layered wound dressing could comprise 1) an inner layer forming the wound contacting surface of the dressing and adhering the dressing to the wound, comprising the biopolymer provided herein, and 2) an outer cover layer/flexible film layer to block penetration of dirt, bacteria, irritants, etc. The multi-layered wound dressing can further comprise additional layer(s) between the inner layer and the outer cover layer. The additional layers can comprise the further additional ingredients discussed herein. The additional layers can also function as additional absorbent layers to further absorb exudate from the wound and provide a reservoir of moisture to deter wound desiccation.

### Uses of Composition and Dressing

In some embodiments, the at least one biopolymer in the composition or dressing helps to absorb moisture and wound extrudate, maintaining a moist environment, and promoting wound healing. In some embodiments, the dressing is a skin dressing, and the biopolymer in the dressing helps to remove excess liquid and perspiration from the skin surface to prevent maceration that can compromise the integrity of skin.

In some embodiments, methods of preparing a dressing comprising at least one biopolymer discussed herein are provided. In some embodiments, the composition comprising the biopolymer(s) may be formulated as a powder, and subsequently rehydrated.

In some embodiments, the compositions are applied to target surface of a wound in a subject. In some embodiments, the compositions are incorporated on or embedded in a dressing, and methods of treating a wound in a subject in need thereof are provided, comprising applying a dressing that comprises the composition comprising a biopolymer provided herein to the surface of the wound. In some embodiments, the dressing is a skin dressing, and methods of treating a skin wound in a subject in need thereof are provided, comprising applying the skin dressing to the subject's skin.

In various embodiments, application of the dressing comprising the at least one biopolymer discussed herein ensures maintenance of a correct degree of moisture in the wound environment at the interface between wound and dressing to promote optimum conditions for rapid and correct healing of the damaged organ, tissue, or skin, such as thermal insulation of the damaged organ, tissue, or skin, absorption of excess exudate and moisture, pain control, protection against exogenous infections, and comfort for the patient. In some embodiments, the improvement in healing to the damaged organ, tissue, or skin is determined at a second time point compared to a first time point that is immediately before the first application of the dressing. In some embodiments, the dressing is replaced three times a day, twice a day, once a day, every other day, every three days, or once per week. In some embodiments, the dressing is applied for a period of one day, two days, three days, one week, two weeks, one month, two months, three months, six months, one year, or longer. By “applied” is meant substantially continuous use of the dressing, replacing the dressing as needed during that time. In some embodiments, the improvement is evident after use of the dressing for one day, three days, five days, one week, two weeks, one month, two months, three months, six months, or one year or more.

### EXAMPLES

### Example 1. Natural Biopolymers Produced by S. meliloti and S. fredii

Naturally occurring biopolymers produced by select Rhizobiaceae strains are shown in FIG. 2. Succinoglycan, also referred to as EPSI, is produced by Sinorhizobium meliloti. The repeating unit consists of a linear main chain of one galactose and three glucose monosaccharides, and a side chain of four glucose molecules. Main chain sugars are linked by β-1,3 and β-1,4 glycosidic bonds. Side chain sugars are linked by β-1,3 and β-1,6 glycosidic bonds. It is acetylated on glucose three of the main chain, succinylated on glucose three of the side chain, and pyruvylated on the terminal glucose of the side chain (Reuber 1993). Galactoglucan, also referred to as EPSII, is also produced by S. meliloti. It is a linear, repeating dimer of galactose and glucose linked by β-1,3 and α-1,3 glycosidic bonds (Glazebrook 1989). Galactose residues are fully pyruvylated, while approximately 70% of glucose units are acetylated (Her 1990). Glucuronoglycan is produced by Sinorhizobium fredii. It consists of a main chain composed of one galactose and five glucose monosaccharides linked by β-1,3, β-1,4 and β-1,6 glycosidic bonds, and a side chain of two glucuronic acids and a terminal galactose linked by α-1,3, α-1,4, and β-1,4 glycosidic bonds. The terminal galactose on the side chain is both acetylated (at carbons 2 and/or 3) and 4,6-pyruvylated (Djordjevic 1986). Glucuronoglycan can be acetylated at a third, unknown location (Rodríquez-Navarro 2014).

### Example 2. Strain Construction

For targeted deletion of selected ORFs, excision of insertion elements, or correction of SNPs, a non-replicating plasmid vector with positive and negative selection markers was used. Since both S. meliloti and S. fredii are amenable to genetic modification using standard molecular biology and strain engineering techniques, this methodology allows for rapid and precise changes to their genomes to create desired genotypes. First, derivatives of the pJQ200SK plasmid (Quandt 1993) carrying deletion cassettes were generated. For deletion cassettes, regions upstream and downstream (usually 500 bp) of the target ORF including start and stop codons were amplified by PCR. For introduction of wild type DNA, regions upstream and downstream of an insertion element were amplified by PCR. Next, plasmids were assembled using the CPEC method (Quan 2009), and sequence verified prior to introduction into S. meliloti. Plasmids were introduced into S. meliloti by tri-parental mating and strains containing single integrations at homologous genomic regions were selected for antibiotic resistance, and verified by PCR using primers outside of amplified regions. Strains positive for integration of plasmids were then streaked to purification, and selected for the ability to grow on sucrose. The presence of the sacB gene on the integrated pJQ200 plasmid causes lethality when strains are grown on sucrose. Strains that are propagated on sucrose will therefore have mutations in sacB itself, or will recombine to “loop out” the integrated plasmid and either revert to wild type or harbor the deleted or modified sequence originally present in the plasmid.

Domesticated strains of S. meliloti, such as strain Rm1021, have lost the ability to produce galactoglucan (Pellock 2002), and there are several genetic modifications that can be introduced to restore this function. These modifications include deletion of the mucR gene, restoration of a wild type allele, expR101, into the expR locus (Gonzalez 1996), or introduction of a wild type expR ORF and promoter (Charoenpanich 2015). These changes all result in an Rm1021-derived strain that produces galactoglucan in addition to succinoglycan. For this study, wild type expR was introduced into strain Rm1021 using the methodology described above. This resulted in strain EXO3, which produces both S. meliloti biopolymers simultaneously.

Strain EXO3 was used to generate derivative strains that produced either succinoglycan or galactoglucan alone, by deleting ORFs that are known to be responsible for the biosynthesis of either exopolysaccharide. For example, a strain that produces succinoglycan can be generated by deletion of any of several glycosyltransferases, including wgaB, or wgeB involved in the synthesis of galactoglucan (Becker 1997). A strain that produces galactoglucan can be generated by deletion of any of several glycosyltransferases, such as exoF, exoA or exoY (Gonzalez 1996, Glazebrook 1989), involved in the initial steps of succinoglycan biosynthesis. Using the techniques described above, the wgeB ORF was deleted to generate an EXO3 derivative only capable of producing succinoglycan. To generate a galactoglucan production strain, the exoY ORF was deleted in EXO3. These strains, EXO1 and EXO2 (Table 1), were used for the subsequent production of succinoglycan or galactoglucan, respectively.

The targeted deletion method described above can be used to generate strains that produce variant biopolymers, such as those that lack chemical modifications. The genes responsible for succinylation and acetylation of succinoglycan, exoH and exoZ, for example, may be deleted from the genome of S. meliloti. To generate a modified version of galactoglucan, wgaE, the gene responsible for pyruvylation was excised from S. meliloti.

TABLE 1
BioPolymer production strains.

| Strain | Species | Genotype | Product |
|---|---|---|---|
| EXO1 | S. meliloti Rm1021 | expR+, ΔwgeB | Succinoglycan |
| EXO2 | S. meliloti Rm1021 | expR+, ΔexoY | Galactoglucan |
| EXO3 | S. meliloti Rm1021 | expR+ | Succinoglycan + galactoglucan |
| EXO19 | S. meliloti Rm1021 | expR+, ΔexoY, ΔwgaE | Galactoglucan (lacking pyruvate) |
| EXO5 | S. fredii NGR234 | Wild type | Glucuronoglycan |

### Example 3. Production and Purification of Biopolymers

For bench scale growth and biopolymer production, batch cultures in shake flasks were used. Production strains were inoculated from culture plates into TY medium and grown overnight in a shaking incubator at 30° C. The next day, the overnight cultures were diluted, typically at a ratio of 1:100 or 1:200, into production medium. Production medium consisted of a defined minimal medium such as M9 containing a carbon source, either glucose or sucrose, at a concentration between 2-4% (w/v), a nitrogen source such as ammonium sulfate, a buffer to maintain neutral pH, divalent cations such as MgSO₄ and CaCl₂, trace elements, and vitamins (U.S. Pat. No. 7,371,558B2). Strains were grown in production medium for up to three days, and then harvested for purification.

Recovery and purification of biopolymers were performed by initial cell separation followed by alcohol precipitation. For high molecular weight polymers, cultures were diluted in either two or three volumes of water and supernatant was separated from cells by centrifugation. For low molecular weight biopolymers it was not necessary to dilute culture broth prior to cell separation. Approximately 1 mM CaCl₂ was then added to the supernatant, and biopolymers were precipitated at room temperature by addition of two volumes of isopropyl alcohol. Precipitates were isolated by centrifugation, and then washed in either 70% or 90% ethyl alcohol. After the wash steps, precipitates were re-isolated by low-speed centrifugation and dried overnight in a 60° C. oven until weight loss stabilized, indicating an absence of residual water. Final product was then ground using a mortar and pestle or using a bench scale mill. Alternatively, following centrifugation, the resulting pellet was redissolved in water. Insoluble material was removed by centrifugation and the product was precipitated as before. The material was dissolved again and treated with proteinase K to digest residual protein, then precipitated a third time. Finally, the pellet was washed with 70% then 100% ethanol and dried at 60° C. The dried product was ground with a mortar and pestle or bench-scale mill.

### Example 4. Characterization of Biopolymers

Biopolymers are characterized by analytical methods. In some instances, NMR spectroscopy may be used to determine structural information on composition, sequence distribution, substitution pattern, and molecular weights. Biopolymers may be assayed by solution-NMR or solid-state NMR. For solution-state NMR of polysaccharides, due to the high viscosity of the material, the sample may be subjected to enzymatic digestion or pretreatment (Her et al. 1990). Samples may be assayed without pre-treatment using solid-state methods, such as ¹³C cross-polarization magic-angle spinning (CPMAS) NMR (Schaefer 1976). More detailed structural analysis and/or quantitation may be assessed by 2D NMR, for example as described in Yao 2021. The person of skill in the art understands that each analytical method has distinct advantages and disadvantages and can select an appropriate analytical method to generate desired information regarding the structure, extent of modification, and/or purity level of biopolymers. Using these methods, the extent of modification of sugars in a polysaccharide chain may be quantified. Levels of acetylation, pyruvylation, succinylation, or other modifying chemical groups, for example, may be determined for a sample of biopolymer.

### Example 5. Water Absorption

To determine the water binding capacity of biopolymers, samples were placed in a sealed, humidified chamber for five days and mass increase was measured in comparison to hyaluronic acid. Biopolymers were purified according to the procedures in Example 3. Prior to conducting the water absorption experiments, biopolymer samples were dried for 30 minutes at 60° C. to ensure that all residual water was evaporated. Small amounts (typically between 25 and 50 mg) were then weighed (value m₀) and placed into individual tared plastic or aluminum trays. All samples were placed on a platform in a sealed plastic chamber containing 250 ml of warm (approximately 35° C.) water. The entire chamber containing all samples was then placed into an incubator at 30° C. After five days, the humidified chamber was opened, and individual samples were weighed (value m) to calculate mass increase. Water binding capacity (WBC) for each sample was calculated according to the following equation: (m−m₀)/m₀. This raw value represents the degree of swelling and can be expressed as percent mass increase by multiplying by 100.

A pure, 5 kDa preparation of hyaluronic acid (HAworks) was used as a control for the water binding experiments. WBC, measured as described above, of hyaluronic acid was typically between 200 and 300%. Higher molecular weights of HA (100 kDa HAworks, and >1000 kDa Acros Organics) were tested and showed similar water absorbing capacities as the low molecular weight sample. To calculate fold change of experimental samples, WBC values for mass increase of biopolymers were normalized to the WBC values for HA within an experiment.

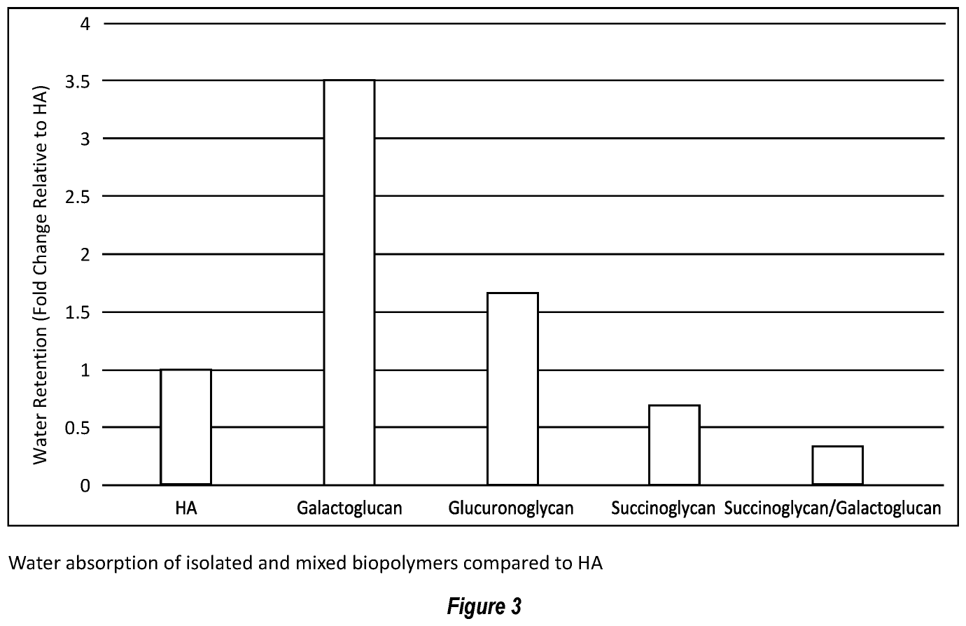

FIG. 3 shows that the WBC of isolated galactoglucan is increased by as much as 3.5-fold in comparison to HA. This WBC value was replicable across multiple experiments. The raw percent increase in mass was 650% for galactoglucan, which was substantially higher than the 185% increase measured for HA. After further incubation in the humidified chamber, the mass increase for galactoglucan reached as high as 720% of its initial mass. Results were similar when galactoglucan was derived from multiple carbon sources including glucose, sucrose, and corn syrup. Glucuronoglycan, isolated from S. fredii, showed a 1.7-fold increase in water binding relative to HA. The raw value for percent mass increase for glucuronoglycan was 311%. Isolated succinoglycan, in contrast to galactoglucan and glucuronoglycan, displayed decreased water binding in comparison to HA (0.7-fold decrease or 129% raw mass increase), and a mixture of succinoglycan and galactoglucan was reduced (0.3-fold decrease or 60% raw mass increase) even further. In the case of galactoglucan, the improved WBC was only observed when galactoglucan was purified independently, and not as the naturally occurring mixture of both galactoglucan and succinoglycan from S. meliloti.

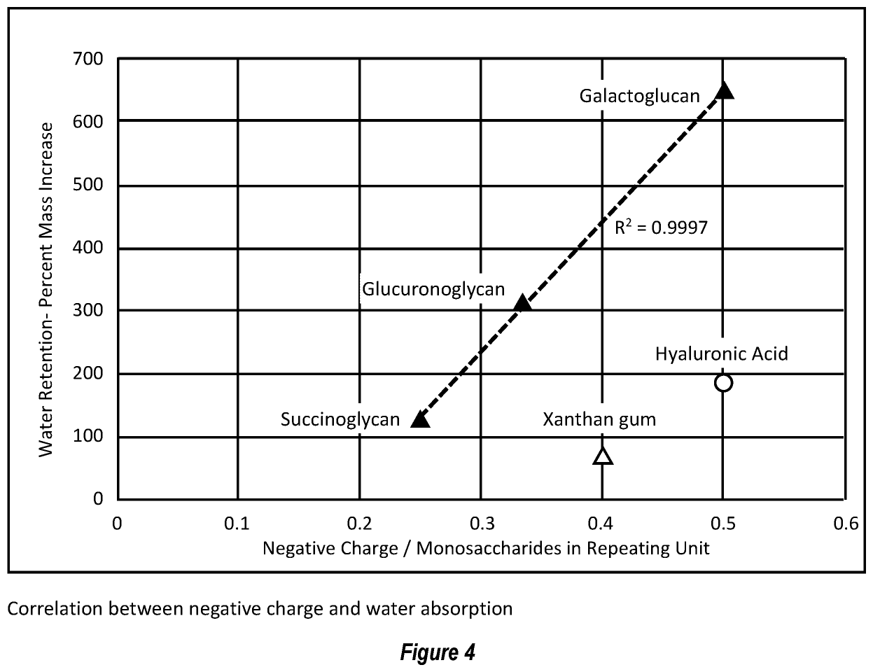

Although monosaccharide type and content, chemical modifications, glycosidic linkages, and molecular weight may all affect the behavior of a biopolymer, for the biopolymers derived from these species of Rhizobiaceae, the degree of negative charge appears to be a predominant factor in WBC. FIG. 4 shows that for the S. meliloti and S. fredii biopolymers, there is a correlation between negative charge and water binding performance. For values on the x-axis, negative charge for each repeating unit was calculated based on the pKₐ of chemical groups or sugar acids at physiological pH. For example, the two glucuronic acids and the pyruvate modification of glucuronoglycan each contribute one negative charge at neutral pH, and therefore the ratio of negative charge to total sugars in the repeating unit is 1:3, or 0.33. For galactoglucan, this ratio is 1:2, and for succinoglycan this ratio is 1:4. These ratios were plotted against the values for percent mass increase of each biopolymer. As shown in FIG. 4, galactoglucan, the molecule with the highest ratio of negative charge to monosaccharides in the repeating unit had the highest capacity for water binding. Other rhizobial biopolymers fit precisely upon this trendline with a high R² value. Hyaluronic acid (gray circle), which also has a charge to monosaccharide ratio of 1:2, did not fit on the trendline, indicating that for this molecule, something other than or in addition to charge ratio affects water retention. Xanthan gum (Modernist Pantry) displayed poor water binding capacity in comparison to HA, and also did not fit on the trendline.

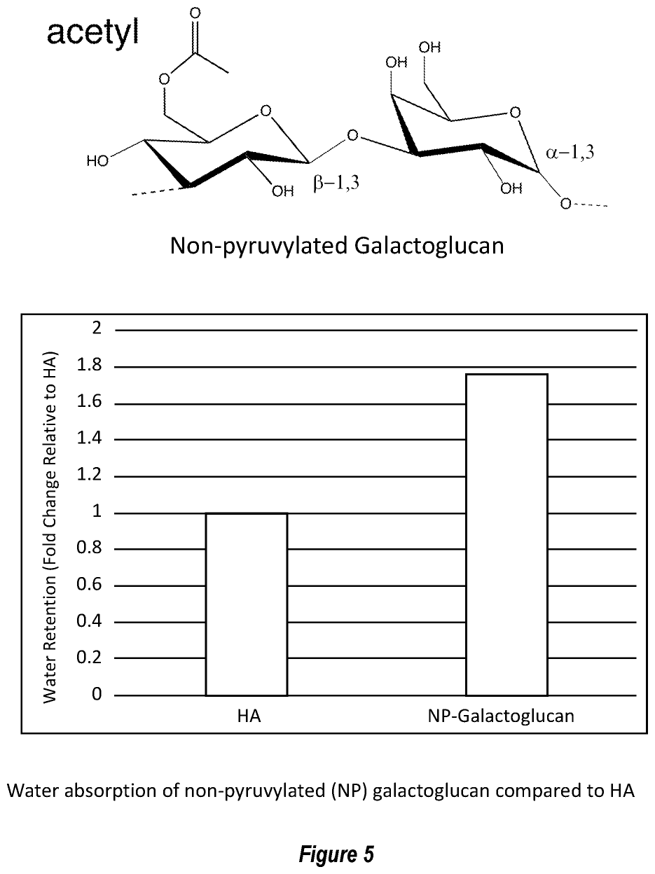

FIG. 5 shows the structure of a non-pyruvylated galactoglucan molecule (NP-galactoglucan), derived from an S. meliloti strain with the wgaE gene excised. The NP-galactoglucan molecule also retained more water than the HA control, although not to the extent of the fully pyruvylated galactoglucan. Compared to HA, the NP-galactoglucan molecule displayed a 1.7-fold increase in the ability to bind water.

### Example 6. Cytotoxicity

To test for cytotoxicity, biopolymers were purified according to Example 3 and resuspended in a Ca-, Mg-free solution of PBS at a concentration of 1% (w/v). These solutions were then heat pasteurized for 30 minutes at 60° C. in a water bath. The cytotoxicity assay described below was carried out at Pacific Biolabs in Hercules, CA.

Test Procedure: A sterile filter paper with a flat surface measuring 1.0 cm² total surface area was saturated with ˜0.1 mL of the test solution and placed directly on the cell culture monolayer in the center of a 10 cm² well. Triplicate preparations were prepared. Triplicate positive and negative controls were tested in the same manner as the test articles. All wells were incubated for not less than 24 hours at 37±1° C. in a humidified incubator with 5±1% CO2. After incubation, the test articles and controls were gently removed from the wells. The cell cultures were examined under an inverted microscope with 100× magnification for cytotoxic response. The response was graded on a scale of 0-4. The achievement of a numerical grade greater than 2 is considered a cytotoxic effect.

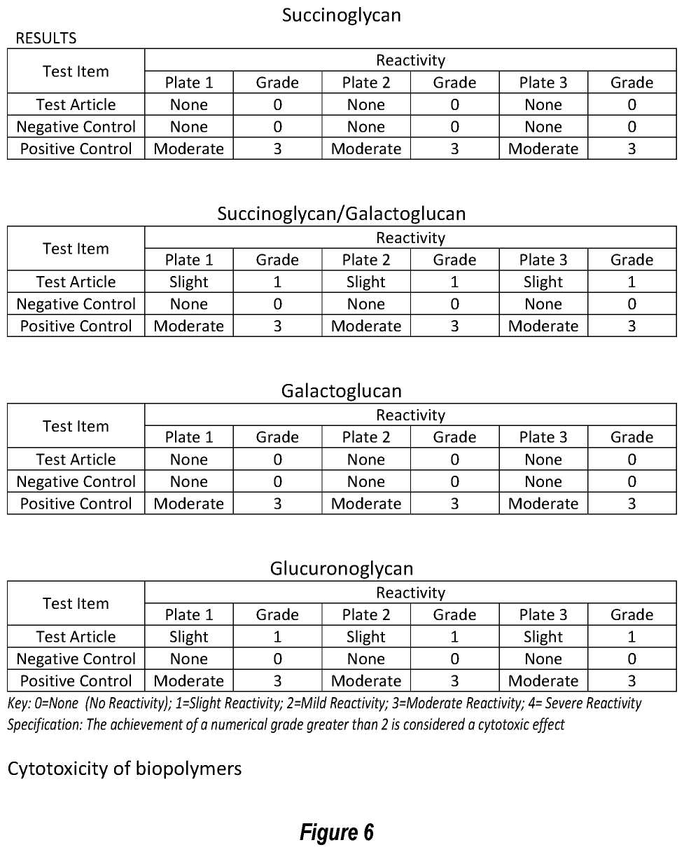

This study was conducted according to ISO 10993-5:2009. A value of 0 is considered no reactivity, and a value of 1 is considered only slightly reactive. FIG. 6 shows that isolated succinoglycan, galactoglucan, glucuronoglycan, and a mixture of succinoglycan and galactoglucan are not cytotoxic according to this assay.

### Example 7. In Vitro Percutaneous Absorption of Biopolymers

Ex vivo dermal studies are used to assess skin penetration and to rank biopolymers in terms of permeability or accumulation, and to optimize formulations for efficacy. Studies are performed with human or porcine skin in Franz diffusion cells (Franz 1975) to assess percutaneous absorption. Biopolymer formulations are applied to the upper (external) surface with the Franz cell, and samples are removed at pre-determined time points from the reservoir containing buffer that is in contact with the lower (serosal) surface, and measured. Control polymers or other compounds are either co-dosed (if the test biopolymer is in solution) or run in parallel (if the test biopolymer is in some other type of formulation) for quality control. The skin may be extracted at the end of the study to quantify accumulation of the test compound.

Franz Cell assays were conducted using non-pyruvylated galactoglucan samples. Specifically, excised human cadaver skin from a single donor was measured for thickness and then mounted in Franz vertical diffusion cells that were thermostatically controlled at 37° C. (FDC-6, Logan instruments, Somerset, NJ). Receptor solution (PBS, pH 7.4) was added, and allowed to equilibrate for 30 minutes at 37° C. (to reach skin surface temperature of 32° C.). After the equilibration period, the entire receptor solution volume (approximately 11 ml) was removed, discarded, and replaced with fresh, pre-warmed receptor solution to remove endogenous background released into the buffer prior to initiation of the experiment.

Dosing solution was prepared by adding 5% (w/v) biopolymer to PBS, pH 7.4 and incubating at 50° C. for 30 minutes. 1 ml of dosing solution was added to each donor chamber. The total amount of biopolymer for each chamber was thus 50 mg. Donor solution was sampled at 0, 8, and 24 hours. Receptor solution was sampled at 0.5, 1, 2, 4, 8, and 24 hrs. After 24 hours, tissue was de-mounted and rinsed briefly in blank PBS after the permeation duration, weighed, and stored individually at −20° C. For quantitation of biopolymer, the anthrone method (Morris 1948) was used in conjunction with the Carrez reagent (EMD Millipore) to reduce protein background in receptor samples. The anthrone method is commonly used to detect the presence of carbohydrate in solution. For receptor samples, 4 μL of Carrez reagent I was added to 400 μL of sample in a 1.5 mL tube and the tube was mixed by vortexing. Another 4 μL of Carrez reagent II was then added and the tube mixed again. The reagent was neutralized with 2.5 μL of 1 N NaOH and tubes were centrifuged 5 minutes at 16,000×g. 625 μL of 0.2% anthrone in concentrated sulfuric acid was mixed with 325 μL of supernatant on ice. Samples were heated 15 minutes at 99° C. then cooled on ice. To detect biopolymer that was retained in the skin, de-mounted samples were soaked in 1 mL PBS in a 2 mL tube at 37° C. for 4 hours then 4° C. for 3 days. The PBS was collected and centrifuged twice at 16,000×g for 5 min to remove debris. The samples were clarified using the Carrez reagent as above then diluted 10-fold in PBS.

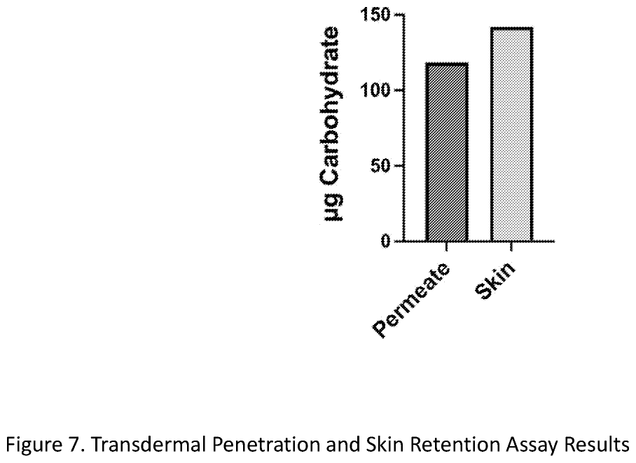

For all samples, absorbance was measured at 620 nm and carbohydrate concentration was determined by comparison to a standard curve that had been clarified using the Carrez reagent. Background absorbance for control Franz Cells (with no biopolymer) was calculated and subtracted from values to derive carbohydrate concentration. FIG. 7 shows representative results from a receptor chamber after four hours of incubation (permeate). Carbohydrate detected in this sample indicates that biopolymer had penetrated the epidermal layer. The highest concentration of biopolymer in the receptor chamber was typically observed at these time points. FIG. 7 also shows that a certain amount of biopolymer was either in or associated with the skin itself (skin) after the de-mounted sample was soaked in PBS as described above. These findings show that a fraction of the material in the donor chamber of the Franz Cell fully permeated the skin samples, and another fraction was retained in or associated with the skin sample. Without intending to be bound by any particular theory, the skin-penetrating fraction of non-pyruvylated galactoglucan may be the lowest molecular weight in the sample, and higher molecular weight fractions may be absorbed by and/or retained upon the surface of the epidermis.

### Example 8. Moisture Absorption into a Matrix

To examine the efficacy of biopolymers on water absorption into a solid matrix, several different methods are used. In some instances, hydrogels may be produced by various chemical methods to make derivatives of biopolymers suitable for crosslinking. Other solid gels may be made that are simply impregnated with biopolymer embedded in a solidified matrix. Gels made of alginate or low melting point agarose are used, for example, for the impregnation method.

Biopolymers are dissolved in PBS at high concentrations (up to 5%), and then those solutions are used to generate solid matrix gels. Gel sections of known mass are then placed in excess PBS solution for 24 h and subsequently weighed to determine the swollen mass. The mass swelling ratio for each biopolymer is then determined as the ratio of the gel (hydrogel or impregnated matrix) equilibrium swelled weight to its initial weight. This measurement reflects the ability of the biopolymer to bind and retain water in an osmotically balanced system representative of a bandage placed upon a wound.

Moisture absorption into an agarose matrix was assessed as follows. Gels were cast by melting 1 g of low melting point agarose in 50 mL of PBS and pouring the molten agarose into a 100 mm diameter petri dish after cooling. In the case of the galactoglucan gel the material was dissolved directly in the gel during cooling. Gels were allowed to set overnight and 1 cm×1 cm cubes were cut with a razor. Cubes were dried overnight at 60° C. and weighed. The cubes were submerged in 5 mL of PBS in a 35 mm petri dish and allowed to swell for one day at room temperature. The cubes were blotted with a Kimwipe to remove surface moisture and weighed to determine swelling capacity, which was calculated as in Example 5.

The results of that experiment are shown in Table 2. Inclusion of 1% galactoglucan in the agarose matrix substantially increased the swelling capacity of the matrix, by about 2-fold.

TABLE 2
Matrix swelling capacity

| Matrix | Swelling capacity^{a} |
|---|---|
| 2% agarose | 281 ± 14 |
| 2% agarose + 1% galactoglucan | 576 ± 2.6 |

^{a}Values are the mean of three replicates ± standard deviation

### Example 9. Fibroblast Migration Assay

Several in vitro methods exist to analyze the effect of different compounds on wound healing. Two of these methods include measuring cell migration after removal of a section of a monolayer (the scratch assay), and fibroblast migration through a collagen matrix (representative of connective tissue in skin).

For the scratch assay, monolayers of primary human keratinocytes or fibroblasts are generated in microtiter plates using standard cell culture growth medium. A section of these cell layers is then removed from the plate surface, and the cells are treated with a solution (typically PBS) containing biopolymer or a control solution and incubated at 37 C under standard conditions for 24 or 48 hours. Cells are then removed from the incubator and washed twice with PBS. The rate of closure of the scratch is then assayed by microscopy. In some cases the production of collagen can be measured by immuno-assay.

We claim:

1. A dressing comprising a biopolymer preparation comprising a biopolymer, wherein the biopolymer is composed of repeating disaccharide units comprising glucose and galactose, wherein at least 60%, at least 65%, at least 70%, at least 75%, at least 80%, at least 85%, or at least 90% of the glucose is acetylated, and wherein at least 40%, at least 45%, at least 50%, at least 55%, at least 60%, at least 65%, at least 70%, at least 75%, at least 80%, at least 85%, at least 90%, at least 95%, or at least 99% of the galactose is pyruvylated, and
wherein the biopolymer preparation is incorporated on or in a solid substrate.

2. The dressing of claim 1, wherein the glucose and galactose are linked by β-1,3 glycosidic bonds and α-1,3 glycosidic bonds.

3. A dressing comprising a biopolymer preparation comprising a biopolymer that is composed of repeating disaccharide units of the structure: 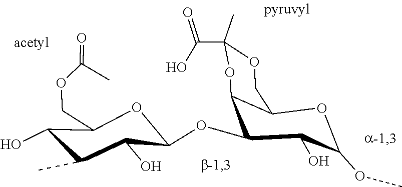
wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the disaccharide units lack the pyruvyl moiety, and wherein the biopolymer preparation is incorporated on or in a solid substrate.

4. The dressing of any one of claims 1-3, wherein the molar ratio of glucose:galactose:pyruvyl:acetyl in the biopolymer is 1:1:0.4-1:0.6-1.

5. The dressing of any one of claims 1-4, wherein the average molecular weight of the biopolymer in the biopolymer preparation is less than 3,000 kDa, less than 1,000 kDa, less than 300 kDa, less than 100 kDa, or less than 40 kDa.

6. The dressing of any one of claims 1-4, wherein the average molecular weight of the biopolymer in the biopolymer preparation is 0.5 kDa to 40 kDa.

7. The dressing of any one of the preceding claims, wherein the biopolymer preparation is capable of absorbing at least the same amount, at least 1.5-fold, at least 2-fold, or at least 3-fold more water than an equal amount of hyaluronic acid.

8. The dressing of any one of the preceding claims, wherein the biopolymer preparation is capable of absorbing an amount of water that is at least 100%, at least 200%, at least 300%, at least 400%, or at least 500% the initial dry weight of the biopolymer preparation.

9. The dressing of claim 7 or claim 8 wherein water absorption is measured by placing a dry sample of the biopolymer preparation in a humidified chamber at 30° C. for five days.

10. The dressing of any one of the preceding claims, wherein the biopolymer preparation is less than 25%, less than 20%, less than 15%, less than 10%, less than 5%, less than 3%, or less than 1% succinoglycan, or is substantially free of succinoglycan.

11. The dressing of any one of the preceding claims, wherein the biopolymer preparation is a powder or rehydrated powder.

12. A dressing comprising a biopolymer preparation comprising a biopolymer that is composed of repeating disaccharide units comprising glucose and galactose, wherein at least 60%, at least 65%, at least 70%, at least 75%, at least 80%, at least 85%, or at least 90% of the glucose is acetylated, and wherein less than 15%, less than 10%, or less than 5% of the galactose is pyruvylated, and wherein the biopolymer preparation is incorporated on or in a solid substrate.

13. The dressing of claim 12, wherein the glucose and galactose are linked by β-1,3 glycosidic bonds and α-1,3 glycosidic bonds.

14. A dressing comprising a biopolymer preparation, wherein the biopolymer is composed of repeating disaccharide units of the structure: 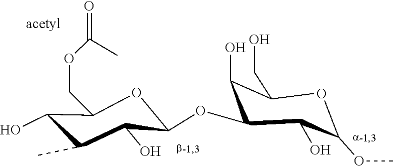
wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 15%, no more than 10%, or no more than 5%, of the disaccharide units are pyruvylated, and wherein the biopolymer preparation is incorporated on or in a solid substrate.

15. The dressing of any one of claims 12-14, wherein the molar ratio of glucose:galactose:pyruvyl:acetyl in the biopolymer is 1:1:<0.5:0.6-1.

16. The dressing of any one of claims 12-15, wherein the average molecular weight of the biopolymer in the biopolymer preparation is less than 3,000 kDa, less than 1,000 kDa, less than 300 kDa, less than 100 kDa, or less than 40 kDa.

17. The dressing of any one of claims 12-15, wherein the average molecular weight of the biopolymer in the biopolymer preparation is 0.5 kDa to 40 kDa.

18. The dressing of any one of claims 12-17, wherein the biopolymer preparation is capable of absorbing at least the same amount, at least 1.1-fold, at least 1.2-fold, at least 1.3-fold, at least 1.4-fold, or at least 1.5-fold more water than an equal amount of hyaluronic acid.

19. The dressing of any one of claims 12-18, wherein the biopolymer preparation is capable of absorbing an amount of water that is at least 100%, at least 200%, at least 300%, or at least 400% the initial dry weight of the biopolymer preparation.

20. The dressing of claim 18 or claim 19, wherein water absorption is measured by placing a dry sample of the biopolymer preparation in a humidified chamber at 30° C. for five days.

21. The dressing of any one of claims 12-20, wherein the biopolymer preparation is less than 25%, less than 20%, less than 15%, less than 10%, less than 5%, less than 3%, or less than 1% succinoglycan.

22. The dressing of any one of claims 12-21, wherein the biopolymer preparation is a powder or rehydrated powder.

23. A dressing comprising a biopolymer preparation comprising a biopolymer that is composed of repeating polysaccharide units of the structure: 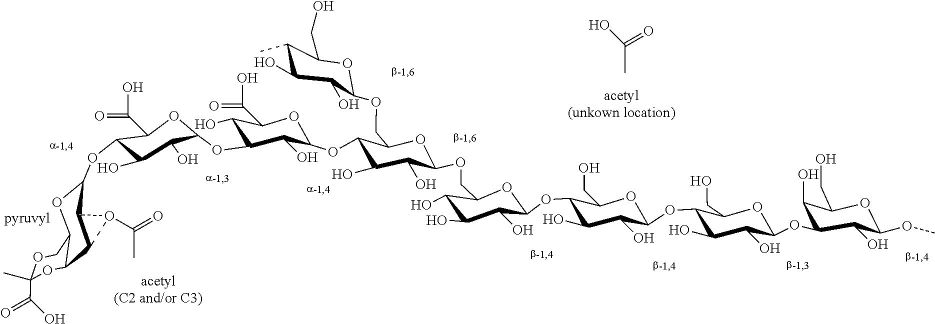
wherein the dotted lines represent the bonds between polysaccharide units; wherein no more than 30%, no more than 25%, no more than 20%, no more than 15%, no more than 10%, no more than 5%, or no more than 1% of the polysaccharide units lack an acetyl moiety; and wherein no more than 10%, no more than 5%, or no more than 1% of the polysaccharide units lack the pyruvyl moiety, and wherein the biopolymer preparation is incorporated on or in a solid substrate.

24. The dressing of claim 23, wherein the average molecular weight of the biopolymer in the biopolymer preparation is less than 3,000 kDa, less than 1,000 kDa, less than 300 kDa, less than 100 kDa, or less than 40 kDa.

25. The dressing of claim 23, wherein the average molecular weight of the biopolymer in the biopolymer preparation is 1.6 kDa to 40 kDa.

26. The dressing of any one of claims 23-25, wherein the biopolymer preparation is capable of absorbing at least the same amount, at least 1.1-fold, at least 1.2-fold, at least 1.3-fold, at least 1.4-fold, or at least 1.5-fold more water than an equal amount of hyaluronic acid.

27. The dressing of any one of claims 23-26, wherein the biopolymer preparation is capable of absorbing an amount of water that is at least 100%, at least 200%, at least 300%, or at least 400% the initial dry weight of the biopolymer preparation.

28. The dressing of claim 26 or claim 27, wherein water absorption is measured by placing a dry sample of the biopolymer preparation in a humidified chamber at 30° C. for five days.

29. The dressing of any one of claims 23-28, wherein the biopolymer preparation is a powder or rehydrated powder.

30. A dressing comprising a biopolymer preparation comprising a biopolymer that is composed of repeating polysaccharide units, wherein each polysaccharide unit comprises 2-15 or 2-12 or 2-10 monosaccharides, and wherein the biopolymer has a negative charge:monosaccharide ratio in the repeating polysaccharide unit of at least 0.3, or at least 0.35, or at least 0.4, or at least 0.45, and wherein the biopolymer preparation is incorporated on or in a solid substrate.

31. The dressing of claim 30, wherein the polysaccharide unit comprises at least one galactose linked to at least one glucose.

32. The dressing of claim 31, wherein at least one galactose is linked to a glucose through a β-1,3 glycosidic bond.

33. The dressing of any one of claims 30-32, wherein the average molecular weight of the biopolymer in the biopolymer preparation is less than 3,000 kDa, less than 1,000 kDa, less than 300 kDa, less than 100 kDa, or less than 40 kDa.

34. The dressing of any one of claims 30-32, wherein the average molecular weight of the biopolymer in the biopolymer preparation is 0.5 kDa to 40 kDa.

35. The dressing of any one of claims 30-34, wherein the biopolymer preparation is capable of absorbing at least the same amount, at least 1.1-fold, at least 1.5-fold, at least 2-fold, or at least 3-fold more water than an equal amount of hyaluronic acid.

36. The dressing of any one of claims 30-35, wherein the biopolymer preparation is capable of absorbing an amount of water that is at least 100%, at least 200%, at least 300%, or at least 400% the initial dry weight of the biopolymer preparation.

37. The dressing of claim 35 or claim 36, wherein water absorption is measured by placing a dry sample of the biopolymer preparation in a humidified chamber at 30° C. for five days.

38. The dressing of any one of claims 30-37, wherein the biopolymer preparation is a powder or rehydrated powder.

39. The dressing of any one of the preceding claims, wherein the dressing is a wound dressing.

40. The dressing of any one of the preceding claims, wherein the dressing is skin dressing.

41. The dressing of any one of the preceding claims, wherein the dressing is a bandage.

42. The dressing of any one of the preceding claims, wherein the biopolymer preparation forms one layer of the dressing.

43. The dressing of any one of the preceding claims, wherein the solid substrate comprises woven materials of natural or synthetic fibers or synthetic polymeric sheets.

44. The dressing of any one of the preceding claims further comprising an adhesive material.

45. The dressing of any one of the preceding claims further comprising one or more additional ingredients selected from the group consisting of an additional hydrocolloid agent, alcohol, peroxide, betadine, sequestration agents, antimicrobials, antibacterials, antivirals, antifungals, anti-inflammatories, wound healing promoters, conditional agents, surfactants, anti-scaring medications, analgesics, anesthetics, and steroids.

46. The dressing of claim 45, wherein the hydrocolloid agents are selected from the group consisting of collagen, cellulose and cellulose derivatives, glycosaminoglycans, pectins, gum arabic, galactomannans, agar, carrageen, alginates, gelatin, caseinates, xanthans, dextrans, scleroglucans, and combinations thereof.

47. The dressing of claim 45 or claim 46, wherein the dressing comprises hyaluronic acid.

48. The dressing of claim 45 or claim 46, wherein the dressing does not comprise hyaluronic acid.

49. The dressing of any one of claims 45-48, wherein the dressing comprises silver.

50. The dressing of any one of claims 45-49, wherein the one or more additional ingredients are incorporated into the layer comprising the biopolymer preparation, into the adhesive material, or into one or more additional layers.

51. The dressing of any one of the preceding claims, wherein the biopolymer preparation is dispersed throughout a hydrophilic polyurethane foam matrix, and the hydrophilic polyurethane foam matrix is incorporated on or in the solid substrate.

52. The dressing of any one of the preceding claims, wherein the dressing is a multi-layered dressing.

53. A method of treating a wound in a subject in need thereof, comprising applying the dressing of any one of the preceding claims to the wound.

54. The method of claim 53, wherein the wound comprises an acute wound, a chronic wound, a necrotizing wound, or an infected wound.

55. The method of claim 53 or claim 54, wherein the wound is a mechanical wound, a thermal wound, a chemical wound, or a radiation wound.

56. The method of any one of claims 53-55, wherein the dressing is applied to the face, ears, forehead, neck, arms, upper chest, legs, feet, and/or hands of the subject.

57. The method of any of claims 53-56, wherein the dressing promotes thermal insulation of damaged organ, tissue, or skin, absorption of excess exudate and moisture, protection against exogenous infections, and optimum moisture in the wound environment at the interface between wound and dressing.

58. A method of treating a wound in a subject in need thereof, comprising applying a composition to the wound, wherein the composition comprises a biopolymer, wherein the biopolymer is composed of repeating disaccharide units comprising glucose and galactose, wherein at least 60%, at least 65%, at least 70%, at least 75%, at least 80%, at least 85%, or at least 90% of the glucose is acetylated, and wherein at least 40%, at least 45%, at least 50%, at least 55%, at least 60%, at least 65%, at least 70%, at least 75%, at least 80%, at least 85%, at least 90%, at least 95%, or at least 99% of the galactose is pyruvylated.

59. The method of claim 58, wherein the glucose and galactose are linked by β-1,3 glycosidic bonds and α-1,3 glycosidic bonds.

60. A method of treating a wound in a subject in need thereof, comprising applying a composition to the wound, wherein the composition comprises a biopolymer, wherein the biopolymer is composed of repeating disaccharide units of the structure: 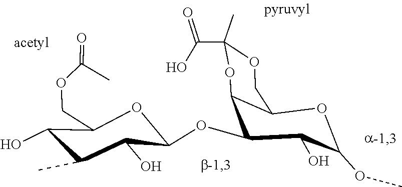
wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the disaccharide units lack the pyruvyl moiety.

61. The method of any one of claims 58-60, wherein the molar ratio of glucose:galactose:pyruvyl:acetyl in the biopolymer is 1:1:0.4-1:0.6-1.

62. The method of any one of claims 58-61, wherein the average molecular weight of the biopolymer in the composition is less than 3,000 kDa, less than 1,000 kDa, less than 300 kDa, less than 100 kDa, or less than 40 kDa.

63. The method of any one of claims 58-61, wherein the average molecular weight of the biopolymer in the composition is 0.5 kDa to 40 kDa.

64. The method of any one of claims 58-63, wherein the biopolymer is capable of absorbing at least the same amount, at least 1.5-fold, at least 2-fold, or at least 3-fold more water than an equal amount of hyaluronic acid.

65. The method of any one of claims 58-64, wherein the biopolymer is capable of absorbing an amount of water that is at least 100%, at least 200%, at least 300%, at least 400%, or at least 500% the initial dry weight of the biopolymer.

66. The method of claim 64 or claim 65, wherein water absorption is measured by placing a dry sample of the biopolymer in a humidified chamber at 30° C. for five days.

67. The method of any one of claims 58-66, which is substantially free of succinoglycan.

68. A method of treating a wound in a subject in need thereof, comprising applying a composition to the wound, wherein the composition comprises a biopolymer, wherein the biopolymer is composed of repeating disaccharide units comprising glucose and galactose, wherein at least 60%, at least 65%, at least 70%, at least 75%, at least 80%, at least 85%, or at least 90% of the glucose is acetylated, and wherein less than 15%, less than 10%, or less than 5% of the galactose is pyruvylated.

69. The method of claim 68, wherein the glucose and galactose are linked by β-1,3 linkages and α-1,3 linkages.

70. A method of treating a wound in a subject in need thereof, comprising applying a composition to the wound, wherein the composition comprises a biopolymer, wherein the biopolymer is composed of repeating disaccharide units of the structure: 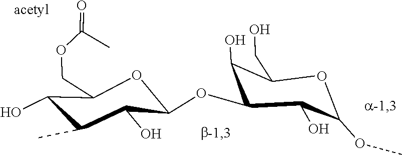
wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 15%, no more than 10%, or no more than 5%, of the disaccharide units are pyruvylated.

71. The method of any one of claims 68-70, wherein the molar ratio of glucose:galactose:pyruvyl:acetyl in the biopolymer is 1:1:<0.5:0.6-1.

72. The method of any one of claims 68-71, wherein the average molecular weight of the biopolymer in the composition is less than 3,000 kDa, less than 1,000 kDa, less than 300 kDa, less than 100 kDa, or less than 40 kDa.

73. The method of any one of claims 68-71, wherein the average molecular weight of the biopolymer in the composition is 0.5 kDa to 40 kDa.

74. The method of any one of claims 68-73, wherein the composition is capable of absorbing at least the same amount, at least 1.1-fold, at least 1.2-fold, at least 1.3-fold, at least 1.4-fold, or at least 1.5-fold more water than an equal amount of hyaluronic acid.

75. The method of any one of claims 68-74, wherein the composition is capable absorbing an amount of water that is at least 100%, at least 200%, at least 300%, or at least 400% the initial dry weight of the biopolymer.

76. The method of claim 74 or claim 75, wherein water absorption is measured by placing a dry sample of the biopolymer in a humidified chamber at 30° C. for five days.

77. The method of any one of claims 68-76, wherein the composition comprises the biopolymer preparation of any one of claims 12-22.

78. The method of any one of claims 68-77, which is substantially free of succinoglycan.

79. A method of treating a wound in a subject in need thereof, comprising applying a composition to the wound, wherein the composition comprises a biopolymer, wherein the biopolymer is composed of repeating polysaccharide units of the structure: 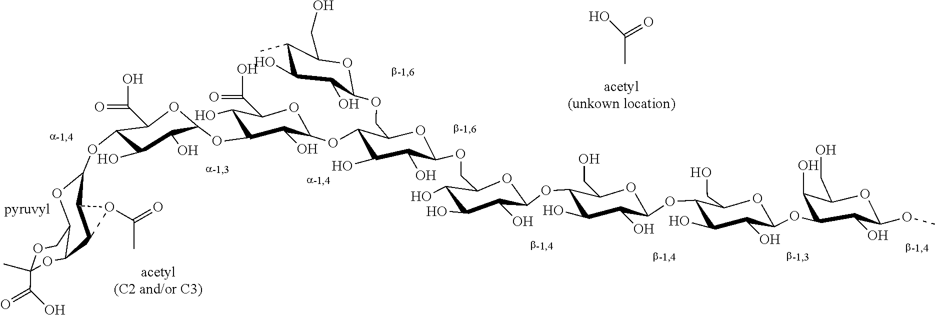
wherein the dotted lines represent the bonds between polysaccharide units; wherein no more than 30%, no more than 25%, no more than 20%, no more than 15%, no more than 10%, no more than 5%, or no more than 1% of the polysaccharide units lack an acetyl moiety; and wherein no more than 10%, no more than 5%, or no more than 1% of the polysaccharide units lack the pyruvyl moiety.

80. The method of claim 79, wherein the average molecular weight of the biopolymer in the composition is less than 3,000 kDa, less than 1,000 kDa, less than 300 kDa, less than 100 kDa, or less than 40 kDa.

81. The method of claim 79, wherein the average molecular weight of the biopolymer in the composition is 1.6 kDa to 40 kDa.

82. The method of any one of claims 79-81, wherein the composition is capable of absorbing at least the same amount, at least 1.1-fold, at least 1.2-fold, at least 1.3-fold, at least 1.4-fold, or at least 1.5-fold more water than an equal amount of hyaluronic acid.

83. The method of any one of claims 79-82, wherein the composition is capable of absorbing an amount of water that is at least 100%, at least 200%, at least 300%, or at least 400% the initial dry weight of the biopolymer.

84. The method of claim 82 or claim 83, wherein water absorption is measured by placing a dry sample of the biopolymer in a humidified chamber at 30° C. for five days.

85. The method of any one of claims 79-84, wherein the composition comprises the biopolymer preparation of any one of claims 23-39.

86. A method of treating a wound in a subject in need thereof, comprising applying a composition to the wound, wherein the composition comprises a biopolymer, wherein the biopolymer is composed of repeating polysaccharide units, wherein each polysaccharide unit comprises 2-15 or 2-12 or 2-10 monosaccharides, and wherein the biopolymer has a negative charge:monosaccharide ratio in the repeating polysaccharide unit of at least 0.3, or at least 0.35, or at least 0.4, or at least 0.45.

87. The method of claim 86, wherein the polysaccharide unit comprises at least one galactose linked to at least one glucose.

88. The method of claim 87, wherein at least one galactose is linked to a glucose through a β1,3 glycosidic bond.

89. The method of any one of claims 86-88, wherein the average molecular weight of the biopolymer in the composition is less than 3,000 kDa, less than 1,000 kDa, less than 300 kDa, less than 100 kDa, or less than 40 kDa.

90. The method of any one of claims 86-88, wherein the average molecular weight of the biopolymer in the composition is 0.5 kDa to 40 kDa.

91. The method of any one of claims 86-90, wherein the composition is capable of absorbing at least the same amount, at least 1.1-fold, at least 1.5-fold, at least 2-fold, or at least 3-fold more water than an equal amount of hyaluronic acid.

92. The method of any one of claims 86-91, wherein the composition is capable of absorbing an amount of water that is at least equal to, at least 1.5 times, at least two times, at least 2.5 times, at least three times, at least four times, or at least five times the initial dry weight of the biopolymer.

93. The method of claim 91 or claim 92, wherein water absorption is measured by placing a dry sample of the biopolymer in a humidified chamber at 30° C. for five days.

94. The method of any one of claims 86-93, wherein the composition comprises the biopolymer preparation of any one of claims 30-38.

95. The method of any one of claims 58-94, wherein the composition further comprising one or more additional ingredients selected from the group consisting of an additional hydrocolloid agent, alcohol, peroxide, betadine, sequestration agents, antimicrobials, antibacterials, antivirals, antifungals, anti-inflammatories, wound healing promoters, conditional agents, surfactants, anti-scaring medications, analgesics, anesthetics, and steroids.

96. The method of claim 95, wherein the hydrocolloid agents are selected from the group consisting of collagen, cellulose and cellulose derivatives, glycosaminoglycans, pectins, gum arabic, galactomannans, agar, carrageen, alginates, gelatin, caseinates, xanthans, dextrans, scleroglucans, and combinations thereof.

97. The method of claim 95 or claim 96, wherein the composition comprises hyaluronic acid.

98. The method of claim 95 or claim 96, wherein the composition does not comprise hyaluronic acid.

99. The method of any one of claims 95-98, wherein the composition comprises silver.

100. The method of any one of claims 58-99, wherein the composition is a salve, emulsion, cream, milk, ointment, syndet, solution, serum, gel, spray or aerosol, foam, suspension, lotion, or stick.

101. The method of claim 100, wherein the composition is incorporated on or in a solid substrate in the form of a dressing.

102. The method of claim 101, wherein the solid substrate comprises woven materials of natural or synthetic fibers or synthetic polymeric sheets.

103. The method of any one of claims 101-102, wherein the dressing further comprising an adhesive material.

104. The method of any one of claims 101-103, wherein the dressing is a wound dressing.

105. The method of any one of claims 101-104, wherein the dressing is skin dressing.

106. The method of any one of claims 101-105, wherein the dressing is a bandage.

107. The method of any one of claims 101-106, wherein the composition forms one layer of the dressing.

108. The method of claim 107, wherein the one or more additional ingredients are incorporated into the layer comprising the biopolymer preparation, in the adhesive material, or in one or more additional layers.

109. The method of any one of claims 101-108, wherein the dressing is a multi-layered dressing.

110. The method of claim 109, wherein the multi-layered dressing comprises 1) an inner layer forming the wound contacting surface of the dressing and adhering the dressing to the wound, comprising the biopolymer, and 2) an outer cover layer.

111. The method of claim 110, wherein the multi-layered dressing further comprises additional layer(s) between the inner layer and the outer cover layer.

112. The method of any one of claims 58-111, wherein the wound comprises an acute wound, a chronic wound, a necrotizing wound, or an infected wound.

113. The method of any of claims 58-112, wherein the wound is a mechanical wound, a thermal wound, a chemical wound, or a radiation wound.

114. The method of any of claims 58-113, wherein the dressing promotes thermal insulation of damaged organ, tissue, or skin, absorption of excess exudate and moisture, protection against exogenous infections, and optimum moisture in the wound environment at the interface between wound and dressing.
